(19) United States
(12) Patent Application Publication
(10) Pub. No.: US 2009/0099061 A1
(43) Pub. Date: Apr. 16, 2009

(54) SYNTHESIS OF CAROTENOID ANALOGS OR DERIVATIVES WITH IMPROVED ANTIOXIDANT CHARACTERISTICS

(76) Inventors: Bente J. Foss, Leira (NO); Geoffry T. Nadolski, Leira (NO); Samuel F. Lockwood, Lake Linden, MI (US)

Correspondence Address: MEYERTONS, HOOD, KIVLIN, KOWERT & GOETZEL, P.C., P.O. BOX 398, AUSTIN, TX, 78767-0398, US

(21) Appl. No.: 12/120,039

(22) Filed: May 13, 2008

Related U.S. Application Data
(63) Continuation-in-part of application No. 12/116,082, filed on May 6, 2008, now abandoned.
Application No. 12/116,082 is a continuation-in-part of application No. PCT/US07/61241, filed on Jan. 29, 2007.
(60) Provisional application No. 60/762,753, filed on Jan. 27, 2006.
(60) Provisional application No. 60/774,726, filed on Feb. 17, 2006.

(51) Int. Cl. A61K 31/05; C07C 39/21; C07C 47/27; C07C 69/76; A61K 31/11; A61K 38/00; A61K 31/70; A61K 31/661; C07F 9/141; C07D 307/62; C07F 9/655

(52) U.S. Cl. 514/2; 568/729; 568/442; 560/76; 558/194; 549/477; 549/222; 514/733; 514/701; 514/130; 514/23

(57) ABSTRACT

A method is described for synthesizing and administering carotenoid compounds with improved antioxidant characteristics. In some embodiments, extension or improvement of conjugation may be employed in structural modification of carotenoids. In other embodiments, reduction of ring/chain steric hindrance may improve the lambda max, and hence, the overall antioxidant capability, of particular compounds. In other embodiments, introduction and/or increase in synthetic handles for conjugation may improve the stoichiometric ratios of conjugating moieties to the polyene backbone. The methods may be used to improve natural and/or synthetic compounds for medicinal application in the treatment of disease.

### PRIORITY CLAIM

This application is a Continuation-in-Part of U.S. patent application Ser. No. 12/116,082 entitled “SYNTHESIS OF CAROTENOID ANALOGS OR DERIVATIVES WITH IMPROVED ANTIOXIDANT CHARACTERISTICS” filed May 6, 2008. This application is also a Continuation-in-Part of International Application No. PCT/U.S.07/61241 entitled “SYNTHESIS OF CAROTENOID ANALOGS OR DERIVATIVES WITH IMPROVED ANTIOXIDANT CHARACTERISTICS” filed Jan. 29, 2007, which claims priority to U.S. Provisional Patent Application No. 60/762,753 entitled “SYNTHESIS OF CAROTENOID ANALOGS OR DERIVATIVES WITH IMPROVED ANTIOXIDANT CHARACTERISTICS” filed Jan. 27, 2006, and to U.S. Provisional Patent Application No. 60/774,726 entitled “SYNTHESIS OF CAROTENOID ANALOGS OR DERIVATIVES WITH IMPROVED ANTIOXIDANT CHARACTERISTICS” filed Feb. 17, 2006, all of which are incorporated by reference herein.

### BACKGROUND OF THE INVENTION

1. Field of the Invention

The invention generally relates to the fields of medicinal and synthetic chemistry. More specifically, the invention relates to the synthesis and use of carotenoids, naturally occurring and synthetic, including analogs, derivatives, and intermediates.

2. Description of the Relevant Art Carotenoids are a group of natural pigments produced principally by plants, yeast, and microalgae. The family of related compounds now numbers greater than 700 described members, exclusive of Z and E isomers. At least fifty (50) carotenoids have been found in human sera or tissues. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranyl diphosphate molecules produces the parent C₄₀ carbon skeleton. Carotenoids without oxygenated functional groups are called “carotenes”, reflecting their hydrocarbon nature; oxygenated carotenes are known as “xanthophylls.” Cyclization at one or both ends of the molecule yields a variety of end groups (illustrative structures are shown in FIG. 1).

Documented carotenoid functions in nature include light harvesting, photoprotection, and protective and sex-related coloration in microscopic organisms, mammals, and birds, respectively. A relatively recent observation has been the protective role of carotenoids against age-related diseases in humans as part of a complex antioxidant network within cells. This role is dictated by the close relationship between the physicochemical properties of individual carotenoids and their in vivo functions in organisms. The long system of alternating double and single bonds in the central part of the molecule (delocalizing the π-orbital electrons over the entire length of the polyene chain) confers the distinctive molecular shape, chemical reactivity, and light-absorbing properties of carotenoids. Additionally, phenoxy chemical moieties can impart light and energy-absorption capacity, and/or antioxidant bioactivity, as exhibited by flavonoid-based natural pigments (cyanidin, delphinidin), and medicinally relevant polyphenols (resveratrol, tocopherols). Interestingly, some carotenoids, such as dihydroxyisorenieratene (FIG. 1), possess enhanced phenoxy moieties, such that these functionalities are in-conjugation with the carotenoid polyene.

Carotenoids with chiral centers may exist either as the R (rectus) or S (sinister) configurations. As examples, astaxanthin and actinioerythrol (with 2 chiral centers at the 3 and 3′ carbons) may exist as 3 possible stereoisomers: 3S, 3′S; 3R, 3′S and 3S, 3′R (identical meso forms); or 3R, 3′R. The relative proportions of each of the stereoisomers may vary by natural source. For example, Haematococcus pluvialis microalgal meal is 99% 3S, 3′S astaxanthin, and is likely the predominant human evolutionary source of astaxanthin. Krill (3R,3′R) and yeast sources yield different stereoisomer compositions than the microalgal source. Synthetic astaxanthin, produced by large manufacturers such as Hoffmann-LaRoche AG, Buckton Scott (USA), or BASF AG, are provided as defined geometric isomer mixtures of a 1:2:1 stereoisomer mixture (3S,3′S; 3R, 3′S, (meso); 3R, 3′R) of non-esterified (free) astaxanthin. Natural source astaxanthin from salmonid fish is predominantly a single stereoisomer (3S,3′S), but does contain a mixture of geometric isomers. Astaxanthin from the natural source Haematococcus pluvialis may contain nearly 50% Z isomers. As stated above, the Z conformational change may lead to a higher steric interference between the two parts of the carotenoid molecule, rendering it less stable, more reactive, and more susceptible to reactivity at low oxygen tensions. In such a situation, in relation to the all-E form, the Z forms: (1) may be degraded first; (2) may better suppress the attack of cells by reactive oxygen species such as superoxide anion; and (3) may preferentially slow the formation of radicals. Overall, the Z forms may initially be thermodynamically favored to protect the lipophilic portions of the cell and the cell membrane from destruction. It is important to note, however, that the all-E form of astaxanthin, unlike 13-carotene, retains significant oral bioavailability as well as antioxidant capacity in the form of its dihydroxy- and diketo-substitutions on the β-ionone rings, and has been demonstrated to have increased efficacy over β-carotene in most studies. The all-E form of astaxanthin has also been postulated to have the most membrane-stabilizing effect on cells in vivo. Therefore, it is likely that the all-E form of astaxanthin in natural and synthetic mixtures of stereoisomers is also extremely important in antioxidant mechanisms, and may be the form most suitable for particular pharmaceutical preparations.

The antioxidant mechanism(s) of carotenoids, (e.g., astaxanthin), includes singlet oxygen quenching, direct radical scavenging, and lipid peroxidation chain breaking. The polyene chain of the carotenoid absorbs the excited energy of singlet oxygen, effectively stabilizing the energy transfer by delocalization along the chain, and dissipates the energy to the local environment as heat. Transfer of energy from triplet-state chlorophyll (in plants) or other porphyrins and proto-porphyrins (in mammals) to carotenoids occurs much more readily than the alternative energy transfer to oxygen to form the highly reactive and destructive singlet oxygen (¹O₂). Carotenoids may also accept the excitation energy from singlet oxygen if any should be formed in situ, and again dissipate the energy as heat to the local environment. This singlet oxygen quenching ability has significant implications in cardiac ischemia, macular degeneration, porphyria, and other disease states in which production of singlet oxygen has damaging effects. In the physical quenching mechanism, the carotenoid molecule may be regenerated (most frequently), or be lost. Carotenoids are also excellent chain-breaking antioxidants, a mechanism important in inhibiting the peroxidation of lipids. Astaxanthin can donate hydrogen (H) to the unstable polyunsaturated fatty acid (PUFA) radical, stopping the chain reaction. Peroxyl radicals may also, by addition to the polyene chain of carotenoids, be the proximate cause for lipid peroxide chain termination. The appropriate dose of astaxanthin and/or its derivatives has been shown to completely suppress the peroxyl radical chain reaction in liposome systems, and completely inhibit the extent of myocardial damage in canine experimental infarction studies. Astaxanthin shares with vitamin E this dual antioxidant defense system of singlet oxygen quenching and direct radical scavenging, and in most instances (and particularly at low oxygen tension in vivo) is superior to vitamin E as a radical scavenger and physical quencher of singlet oxygen.

Carotenoids, (e.g., astaxanthin), are potent direct radical scavengers and singlet oxygen quenchers and possess all the desirable qualities of such therapeutic agents for inhibition or amelioration of ischemia-reperfusion injury. Synthesis of novel carotenoid derivatives with “soft-drug” properties (e.g., active as antioxidants in the derivatized form), with physiologically relevant, cleavable linkages to pro-moieties, can generate significant levels of free carotenoids in both plasma and solid organs. In the case of non-esterified, free astaxanthin, this is a particularly useful embodiment (characteristics specific to non-esterified, free astaxanthin below):
Lipid soluble in natural form; may be modified to become more water soluble;
Molecular weight of 597 Daltons (size <600 daltons (Da) readily crosses the blood brain barrier, or BBB);
Long polyene chain characteristic of carotenoids effective in singlet oxygen quenching and lipid peroxidation chain breaking; and
No pro-vitamin A activity in mammals (eliminating concerns of hypervitaminosis A and retinoid toxicity in humans).

The administration of antioxidants that are potent singlet oxygen quenchers and direct radical scavengers, particularly of superoxide anion, should limit hepatic fibrosis and the progression to cirrhosis by affecting the activation of hepatic stellate cells early in the fibrogenetic pathway. Reduction in the level of “Reactive Oxygen Species” (ROS) by the administration of a potent antioxidant can therefore be crucial in the prevention of the activation of both “hepatic stellate cells” (HSC) and Kupffer cells. This protective antioxidant effect appears to be spread across the range of potential therapeutic antioxidants, including water-soluble (e.g., vitamin C, glutathione, resveratrol) and lipophilic (e.g., vitamin E, β-carotene, astaxanthin) agents. Therefore, a co-antioxidant derivative strategy in which water-soluble and lipophilic agents are combined synthetically is a particularly useful embodiment. Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” to Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” to Lockwood et al. (International Publication Number WO 2004/011423 A2, published on Feb. 5, 2004) both of which are incorporated by reference as if fully set forth herein.

Vitamin E is generally considered the reference antioxidant. When compared with vitamin E, carotenoids are more efficient in quenching singlet oxygen in homogeneous organic solvents and in liposome systems. They are better chain-breaking antioxidants as well in liposomal systems. They have demonstrated increased efficacy and potency in vivo. They are particularly effective at low oxygen tension, and in low concentration, making them extremely effective agents in disease conditions in which ischemia is an important part of the tissue injury and pathology. These carotenoids also have a natural tropism for the heart and liver after oral administration. Therefore, therapeutic administration of carotenoids should provide a greater benefit in limiting fibrosis than vitamin E.

Problems related to the use of some carotenoids and structural carotenoid analogs or derivatives include: (1) the complex isomeric mixtures, including non-carotenoid contaminants, provided in natural and synthetic sources leading to costly increases in safety and efficacy tests required by such agencies as the FDA; (2) limited bioavailability upon administration to a subject; and (3) the differential induction of cytochrome P450 enzymes (this family of enzymes exhibits species-specific differences which must be taken into account when extrapolating animal work to human studies). Selection of the appropriate analog or derivative and isomer composition for a particular application increases the utility of carotenoid analogs or derivatives for the uses defined herein.

Efficient synthetic routes can provide a stable source of starting materials (e.g., carotenoids), which may be difficult or expensive to extract from natural sources. Synthesizing analogs of naturally occurring carotenoids may allow for the preparation of biologically active analogs possessing enhanced antioxidant characteristics. Extending the conjugated polyene and/or incorporating in-conjugation phenoxy moieties augments the degree or amount of energy a xanthophyll or analog can absorb and dissipate, and may enhance antioxidant and/or anti-inflammatory bioactivities.

### SUMMARY

A synthetic route to a carotenoid, carotenoid analog or derivative and/or synthetic intermediate is presented. In some embodiments, methods and reactions described herein may be used to synthesize naturally-occurring carotenoids. Naturally-occurring carotenoids may include astaxanthin as well as other carotenoids including, but not limited to, actinioerythrol, capsorubin, renierapurpurin, isorenieratene, violerythrin, astacene, zeaxanthin, carotenediol, nostoxanthin, crustaxanthin, canthaxanthin, isozeaxanthin, hydroxycanthaxanthin, tetrahydroxycarotene-dione, lutein, lycophyll, and lycopene.

In some embodiments, a chemical compound may have the structure:

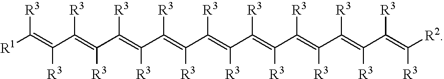

Each R³ may be independently hydrogen or methyl. Each R¹ and R² may be independently:

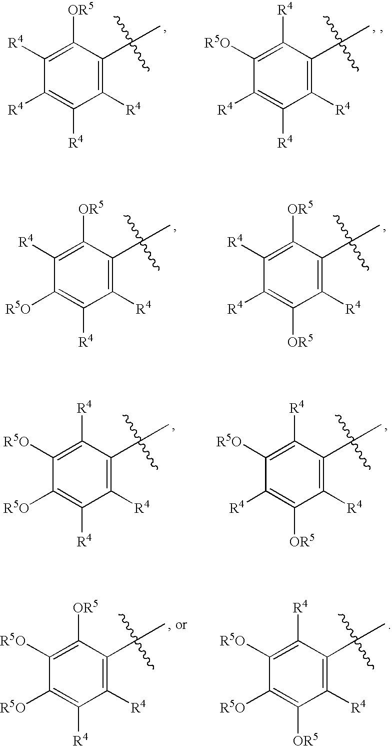

Each R⁴ may be independently hydrogen or methyl. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(R⁶)₃; —C(NR⁶)-aryl-N⁺(R⁶)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. In some embodiments, R⁵ is an amino acid, amino acid derivative, or amino acid analog. In other embodiment, R⁵ is an alkyl amine. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. R⁹ may be a co-antioxidant. In some embodiments, n is 1 to 9. In some embodiments, the chemical compound may form at least a portion of a composition. In some embodiments, a method of inhibiting and/or ameliorating a disease associated with reactive oxygen species and/or other radical and non-radical species may comprise administering to a subject the chemical compound.

In some embodiments, a chemical compound having the structure:

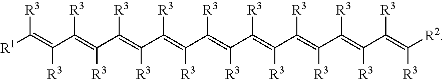

Each R³ may be independently hydrogen or methyl. Each R¹ and R² may be independently:

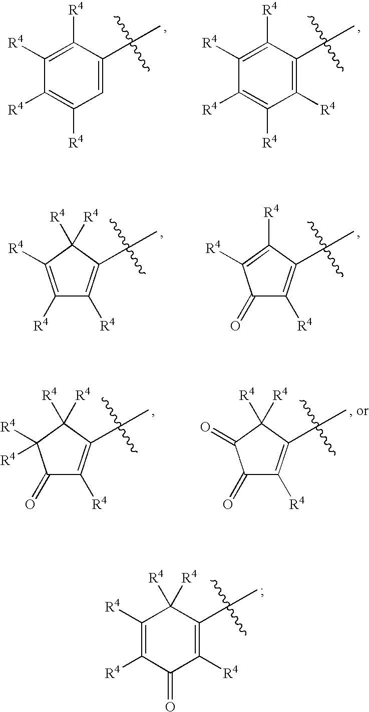

Each R⁴ may be independently hydrogen, methyl, —OH, or —OR⁵. At least one R⁴ group may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(R⁶)₃; —C(NR⁶)-aryl-N⁺(R⁶)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. At least one R⁵ may be -alkyl-N(R⁶)₂; -aryl-N(R⁶)₂; -alkyl-CO₂H; -aryl-CO₂H; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. In some embodiments, n is 1 to 9. In some embodiments, the chemical compound may form at least a portion of a composition. In some embodiments, the chemical compound may form at least a portion of a composition. In some embodiments, a method of inhibiting and/or ameliorating a disease associated with reactive oxygen species and/or other radical and non-radical species may comprise administering to a subject the chemical compound.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

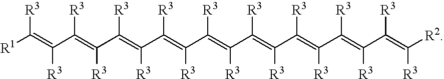

Each R³ may be independently hydrogen or methyl. Each R¹ and R² may be independently:

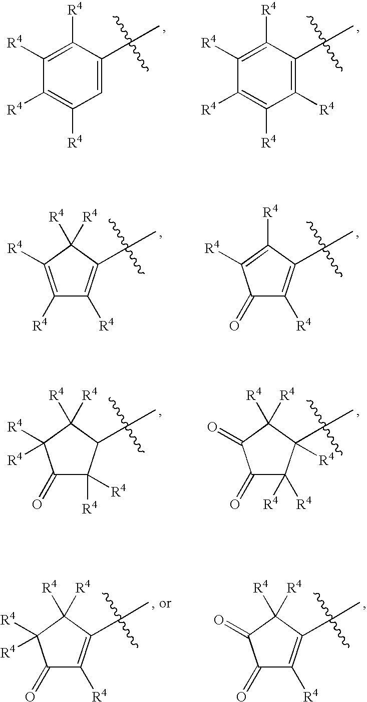

Each R⁴ may be independently hydrogen, methyl, —OH, or —OR⁵. At least one R⁴ group may be —OR⁵. Each R⁵ may be independently: hydrogen, alkyl; aryl; -allyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

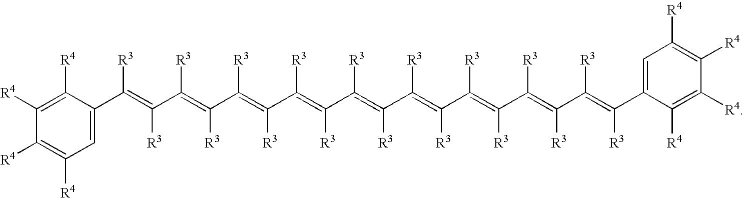

Each R³ may be independently hydrogen or methyl. Each R⁴ may be independently hydrogen, methyl, —OH, or —OR⁵. At least two R⁴ groups may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

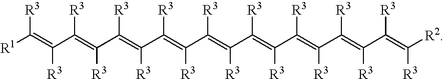

Each R³ may be independently hydrogen or methyl. Each R¹ and R² may be independently:

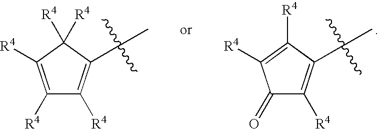

Each R⁴ may be independently hydrogen, methyl, —OH, or —OR⁵. At least one R⁴ group may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

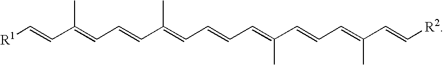

Each R³ may be independently hydrogen or methyl, and where each R¹ and R² may be independently:

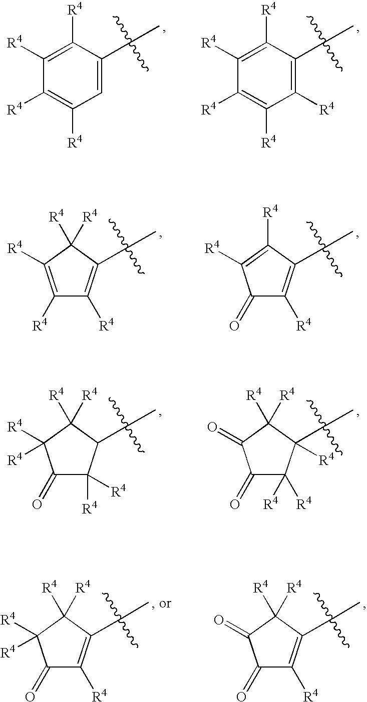

Each R⁴ may be independently hydrogen, methyl, —OH, or —OR⁵. At least one R⁴ group may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂₁; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

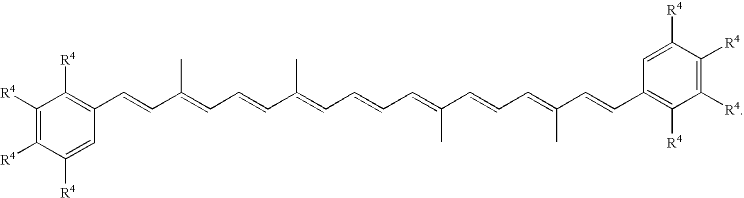

Each R⁴ may be independently hydrogen, methyl, —OH, or —OR⁵. At least one R⁴ group may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

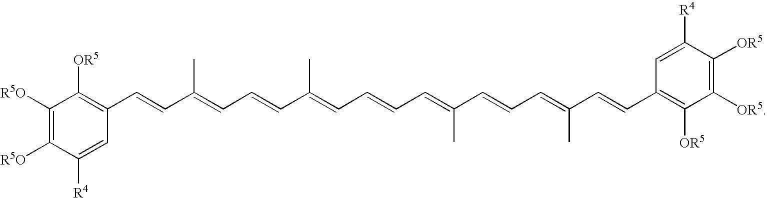

Each R⁴ may be independently hydrogen, methyl, —OH, or —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁵ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

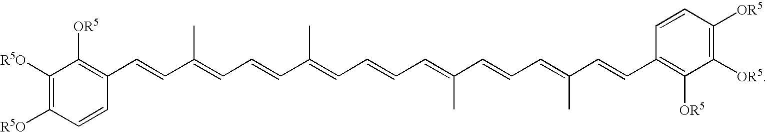

Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may be 1 to 9.

In some embodiments, a substituent —OR⁵ may include:

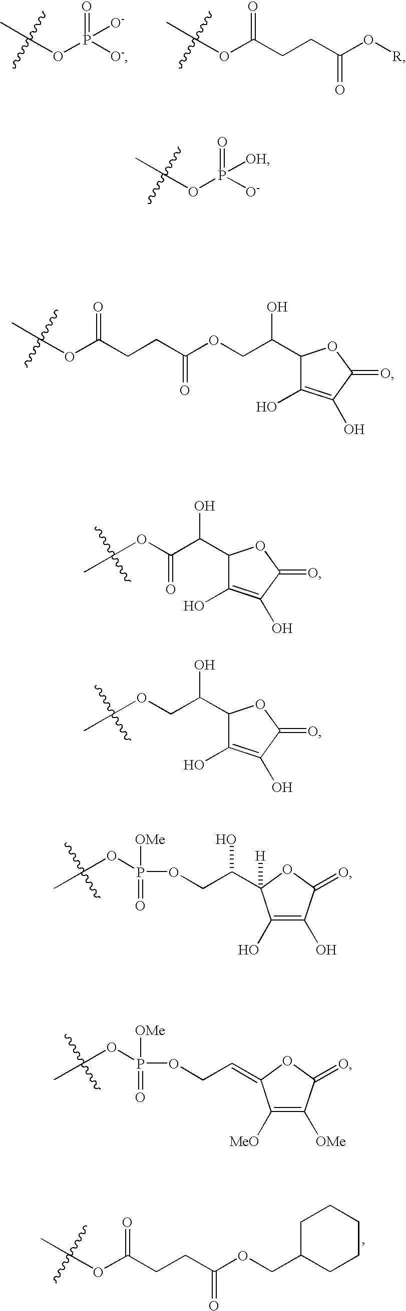

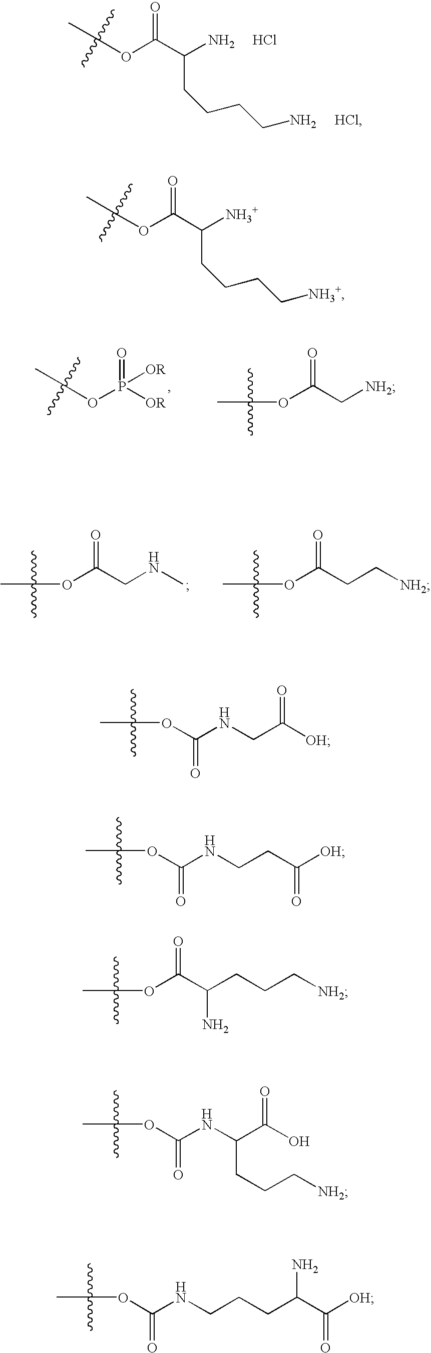

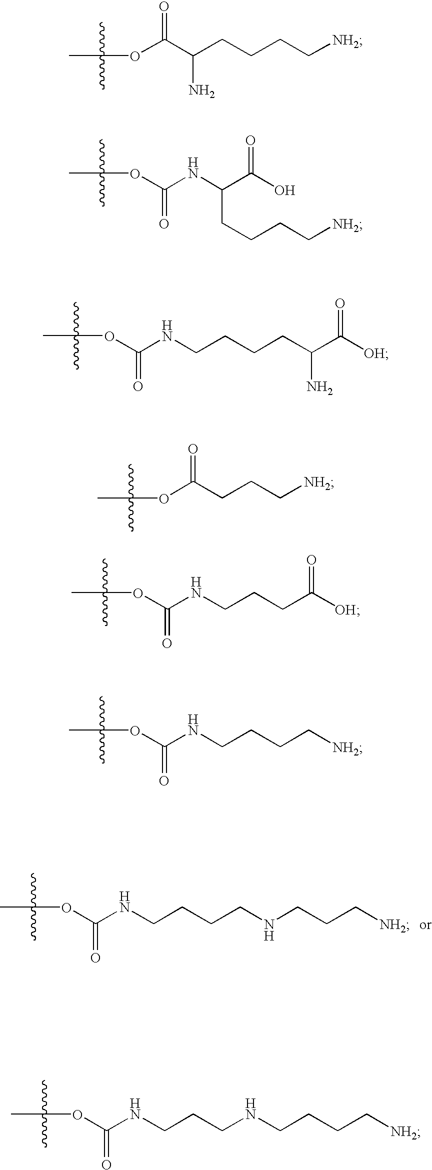

Each R may be independently H, alkyl, aryl, benzyl, Group IA metal, or co-antioxidant.

In some embodiments, a substitutent —OR⁵ may include:

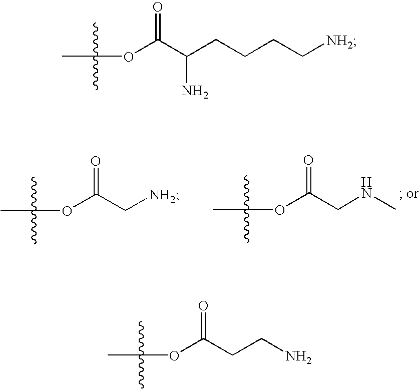

or pharmaceutically acceptable salts thereof.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, polyphenolics, flavonoids, flavonoid analogs, or flavonoid derivatives), or combinations thereof. Flavonoids may include, for example, quercetin, xanthohumol, may beoxanthohumol, or genmay betein; polyphenolics may include, for example, resveratrol.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 depicts a graphic representation of several examples of “parent” carotenoid structures as found in nature.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Unless defined otherwise, all technical and scientific terms used herein have the same meaning as commonly understood by one of ordinary skill in the art.

Compounds described herein embrace both racemic and optically active compounds. Chemical structures depicted herein that do not designate specific stereochemistry are intended to embrace all possible stereochemistries.

It will be appreciated by those skilled in the art that compounds having one or more chiral center(s) may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound. As used herein, the term “single stereoisomer” refers to a compound having one or more chiral center that, while it can exist as two or more stereoisomers, is isolated in greater than about 95% excess of one of the possible stereoisomers. As used herein a compound that has one or more chiral centers is considered to be “optically active” when isolated or used as a single stereoisomer.

The term “acyl” generally refers to a carbonyl substituent, —C(O)R, where R is alkyl or substituted alkyl, aryl, or substituted aryl, which may be called an alkanoyl substituent when R is alkyl.

The terms “alkenyl” and “olefin” generally refer to any structure or moiety having the unsaturation C═C. As used herein, the term “alkynyl” generally refers to any structure or moiety having the unsaturation C≡C.

The term “alkoxy” generally refers to an —OR group, where R is an alkyl, substituted lower alkyl, aryl, substituted aryl. Alkoxy groups include, for example, methoxy, ethoxy, phenoxy, substituted phenoxy, benzyloxy, phenethyloxy, t-butoxy, and others.

The term “alkyl” as used herein generally refers to a chemical substituent containing the monovalent group CₙH₂ₙ, where n is an integer greater than zero. Alkyl includes a branched or unbranched monovalent hydrocarbon radical. An “n-mC” alkyl or “(nC-mC)alkyl” refers to all alkyl groups containing from n to m carbon atoms. For example, a 1-4C alkyl refers to a methyl, ethyl, propyl, or butyl group. All possible isomers of an indicated alkyl are also included. Thus, propyl includes isopropyl, butyl includes n-butyl, isobutyl and t-butyl, and so on. The term alkyl includes substituted alkyls. For example, alkyl includes, but is not limited to: methyl, ethyl, propyl, isopropyl, butyl, iso-butyl, sec-butyl, pentyl, 3-pentyl, hexyl, heptyl, octyl, nonyl, decyl, undecyl, dodecyl, tridecyl, tetradecyl or pentadecyl; “alkenyl” includes but is not limited to vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl; 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, 1-tridecenyl, 2-tridecenyl, 3-tridecenyl, 4-tridecenyl, 5-tridecenyl, 6-tridecenyl, 7-tridecenyl, 8-tridecenyl, 9-tridecenyl, 10-tridecenyl, 11-tridecenyl, 12-tridecenyl, 1-tetradecenyl, 2-tetradecenyl, 3-tetradecenyl, 4-tetradecenyl, 5-tetradecenyl, 6-tetradecenyl, 7-tetradecenyl, 8-tetradecenyl, 9-tetradecenyl, 10-tetradecenyl, 11-tetradecenyl, 12-tetradecenyl, 13-tetradecenyl, 1-pentadecenyl, 2-pentadecenyl, 3-pentadecenyl, 4-pentadecenyl, 5-pentadecenyl, 6-pentadecenyl, 7-pentadecenyl, 8-pentadecenyl, 9-pentadecenyl, 10-pentadecenyl, 11-pentadecenyl, 12-pentadecenyl, 13-pentadecenyl, 14-pentadecenyl; “alkoxy” includes but is not limited to methoxy, ethoxy, propoxy, isopropoxy, butoxy, iso-butoxy, sec-butoxy, pentoxy, 3-pentoxy, hexoxy, heptyloxy, octyloxy, nonyloxy, decyloxy, undecyloxy, dodecyloxy, tridecyloxy, tetradecyloxy, or pentadecyloxy.

The term “amino” generally refers to a group —NRR′, where R and R′ may independently be hydrogen, lower alkyl, substituted lower alkyl, aryl, substituted aryl or acyl.

The terms “amphiphile” or “amphiphilic” refer to a molecule or species, which exhibits both hydrophilic and lipophilic character. In general, an amphiphile contains a lipophilic moiety and a hydrophilic moiety. The terms “lipophilic” and “hydrophobic” are interchangeable as used herein. An amphiphile may form a Langmuir film. An amphiphile may be surface-active in solution. A bolaamphiphile is a special case in which the hydrophobic spacer is substituted on each end with a hydrophilic moiety.

Non-limiting examples of hydrophobic groups or moieties include lower alkyl groups, alkyl groups having 7, 8, 9, 10, 11, 12, or more carbon atoms, including alkyl groups with 14-30, or 30 or more carbon atoms, substituted alkyl groups, alkenyl groups, alkylyl groups, aryl groups, substituted aryl groups, saturated or unsaturated cyclic hydrocarbons, heteroaryl, heteroarylalkyl, heterocyclic, and corresponding substituted groups. A hydrophobic group may contain some hydrophilic groups or substituents insofar as the hydrophobic character of the group is not outweighed. In further variations, a hydrophobic group may include substituted silicon atoms, and may include fluorine atoms. The hydrophobic moieties may be linear, branched, or cyclic.

Non-limiting examples of hydrophilic groups or moieties include hydroxyl, methoxy, phenyl, carboxylic acids and salts thereof, methyl, ethyl, and vinyl esters of carboxylic acids, amides, amino, cyano, isocyano, nitrile, ammonium salts, sulfonium salts, phosphonium salts, mono- and di-alkyl substituted amino groups, polypropyleneglycols, polyethylene glycols, epoxy groups, acrylates, sulfonamides, nitro, —OP(O)(OCH₂CH₂N⁺RRR)O⁻, guanidinium, aminate, acrylamide, pyridinium, piperidine, and combinations thereof, wherein each R is independently selected from H or alkyl. Further examples include polymethylene chains substituted with alcohol, carboxylate, acrylate, or methacrylate. Hydrophilic moieties may also include alkyl chains having internal amino or substituted amino groups, for example, internal —NH—, —NC(O)R—, or —NC(O)CH═CH₂-groups, wherein R is H or alkyl. Hydrophilic moieties may also include polycaprolactones, polycaprolactone diols, poly(acetic acid)s, poly(vinyl acetates)s, poly(2-vinyl pyridine)s, cellulose esters, cellulose hydroxylethers, poly(L-lysine hydrobromide)s, poly(itaconic acid)s, poly(maleic acid)s, poly(styrenesulfonic acid)s, poly(aniline)s, or poly(vinyl phosphonic acid)s. A hydrophilic group may contain some hydrophobic groups or substituents insofar as the hydrophilic character of the group is not outweighed.

The term “antioxidant” as used herein generally refers to any of various substances (e.g., beta-carotene, vitamin C, vitamin E, flavonoids, polyphenolics, and alpha-tocopherol) that inhibit oxidation or reactions promoted by oxygen and peroxides and that include many held to protect the living body from the deleterious effects of free radicals.

The term “aryl” as used herein generally refers to a chemical substituent containing an aromatic group. An aromatic group may be a single aromatic ring or multiple aromatic rings that are fused together, coupled covalently, or coupled to a common group such as a methylene, ethylene, or carbonyl, and includes polynuclear ring structures. An aromatic ring or rings may include, but is not limited to, substituted or unsubstituted phenyl, naphthyl, biphenyl, diphenylmethyl, and benzophenone groups. The term “aryl” includes substituted aryls.

The term “co-antioxidant” as used herein generally refers to an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative potential of one or more anti-oxidants acting synergistically may be greater than the sum of the oxidative potential of each component anti-oxidant).

The terms “coupling” and “coupled” with respect to molecular moieties or species, atoms, synthons, cyclic compounds, and nanoparticles refers to their attachment or association with other molecular moieties or species, atoms, synthons, cyclic compounds, and nanoparticles. The attachment or association may be specific or non-specific, reversible or non-reversible, the result of chemical reaction, or complexation or charge transfer. The bonds formed by a coupling reaction are often covalent bonds, or polar-covalent bonds, or mixed ionic-covalent bonds, and may sometimes be Coulombic forces, ionic or electrostatic forces or interactions.

The term “cycloalkyl” includes, but is not limited to, cyclopropyl, cyclobutyl, cyclopentyl, cyclohexyl, cycloheptyl, or cyclooctyl.

The term “functionalized” as used herein generally refers to the presence of a reactive chemical moiety or functionality. A functional group may include, but is not limited to, chemical groups, biochemical groups, organic groups, inorganic groups, organometallic groups, aryl groups, heteroaryl groups, cyclic hydrocarbon groups, amino (—NH₂), hydroxyl (—OH), cyano (—C≡N), nitro (NO₂), carboxyl (—COOH), formyl (—CHO), keto (—CH₂C(O)CH₂—), ether (—CH₂—O—CH₂—), thioether (—CH₂—S—CH₂—), alkenyl (—C═C—), alkynyl, (—C≡C—), epoxy (e.g.

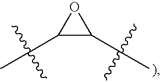

metalloids (functionality containing Si and/or B) and halo (F, Cl, Br, and I) groups. In some embodiments, the functional group is an organic group.

The term “heteroaryl” generally refers to a completely unsaturated heterocycle.

The term “heterocycle” as used herein generally refers to a closed-ring structure, in which one or more of the atoms in the ring is an element other than carbon. Heterocycle may include aromatic compounds or non-aromatic compounds. Heterocycles may include rings such as thiophene, pyridine, isoxazole, phthalimide, pyrazole, indole, furan, or benzo-fused analogs of these rings. Examples of heterocycles include tetrahydrofuran, morpholine, piperidine, pyrrolidine, and others. In some embodiments, “heterocycle” is intended to mean a stable 5- to 7-membered monocyclic or bicyclic or 7- to 10-membered bicyclic heterocyclic ring which is either saturated or unsaturated, and which consists of carbon atoms and from 1 to 4 heteroatoms (e.g., N, O, and S) and wherein the nitrogen and sulfur heteroatoms may optionally be oxidized, and the nitrogen may optionally be quaternized, and including any bicyclic group in which any of the above-defined heterocyclic rings is fused to a benzene ring. In some embodiments, heterocycles may include cyclic rings including boron atoms. The heterocyclic ring may be attached to its pendant group at any heteroatom or carbon atom that results in a stable structure. The heterocyclic rings described herein may be substituted on carbon or on a nitrogen atom if the resulting compound is stable. Examples of such heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzofuranyl, benzothiophenyl, carbazole, chromanyl, chromenyl, cinnolinyl, decahydroquinolinyl, furanyl, furazanyl, imidazolidinyl, imidazolinyl, imidazolyl, indolinyl, indolizinyl, indolyl, isobenzofuranyl, isochromanyl, isoindolinyl, isoindolyl, isoquinolinyl (benzimidazolyl), isothiazolyl, isoxazolyl, morpholinyl, naphthyridinyl, octahydroisoquinolinyl, oxazolidinyl, oxazolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, pyrrolyl, quinazolinyl, quinolinyl, quinoxalinyl, quinuclidinyl, carbolinyl, tetrahydrofuranyl, tetrahydroisoquinolinyl, tetrahydroquinolinyl, tetrazolyl, thianthrenyl, thiazolyl, thienyl, thiophenyl, triazinyl, xanthenyl. Also included are fused ring and spiro compounds containing, for example, the above heterocycles.

The term “ion” as used herein generally refers to an atom(s), radical, or molecule(s) that has lost or gained one or more electrons and has thus acquired an electric charge.

The term “microbe” as used herein generally refers to a minute life form; a microorganism. In some embodiments, a microbe may include a bacterium that causes disease.

The terms “oligomeric” and “polymeric” are used interchangeably herein to generally refer to multimeric structures having more than one component monomer or subunit.

The term “pharmaceutically acceptable salts” includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The term “polymerizable element,” as used herein, generally refers to a chemical substituent or moiety capable of undergoing a self-polymerization and/or co-polymerization reaction (e.g., vinyl derivatives, butadienes, trienes, tetraenes, diolefins, acetylenes, diacetylenes, styrene derivatives).

The terms “Rⁿ” in a chemical formula refer to hydrogen or a functional group, each independently selected, unless stated otherwise. In some embodiments the functional group may be an organic group. In some embodiments the functional group may be an alkyl group. In some embodiment, the functional group may be a hydrophobic or hydrophilic group.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “substituted alkyl” generally refers to an alkyl group with an additional group or groups attached to any carbon of the alkyl group. Substituent groups may include one or more functional groups such as alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbons, heterocycles, and other organic groups.

The term “substituted aryl” generally refers to an aryl group with an additional group or groups attached to any carbon of the aryl group. Additional groups may include one or more functional groups such as lower alkyl, aryl, acyl, halogen, alkylhalo, hydroxy, amino, alkoxy, alkylamino, acylamino, acyloxy, aryloxy, aryloxyalkyl, thioether, heterocycles, both saturated and unsaturated cyclic hydrocarbons which are fused to the aromatic ring(s), coupled covalently or coupled to a common group such as a methylene or ethylene group, or a carbonyl coupling group such as in cyclohexyl phenyl ketone, and others.

The term “substituted heterocycle” generally refers to a heterocyclic group with an additional group or groups attached to any element of the heterocyclic group. Additional groups may include one or more functional groups such as lower alkyl, aryl, acyl, halogen, alkylhalos, hydroxy, amino, alkoxy, alkylamino, acylamino, acyloxy, aryloxy, aryloxyalkyl, thioether, heterocycles, both saturated and unsaturated cyclic hydrocarbons which are fused to the heterocyclic ring(s), coupled covalently or coupled to a common group such as a methylene or ethylene group, or a carbonyl coupling group such as in cyclohexyl phenyl ketone, and others.

The term “substrate” generally refers to a body or base layer or material (e.g., onto which other layers are deposited).

The term “thioether” generally refers to the general structure R—S—R′ in which R and R′ are the same or different and may be alkyl, aryl or heterocyclic groups. The group —SH may also be referred to as “sulfhydryl” or “thiol” or “mercapto.”

The theoretical maximum absorbance for a linear polyene of infinite length is approximately 608 μm [Vetter et al. 1971 (Isler)]. Modifications to the chromophore of polyenic compounds such as carotenoids can be accomplished, while at the same time preserving the co-evolution of absolute carotenoid length with the mammalian plasma, and other, cellular membranes.

Symmetric and non-symmetric ends of these molecules may contain polar (e.g. oxygen) substitutions, which may maintain a perpendicular or moderately angled orientation in the mammalian membrane. The orientation is non-random, with the polar end groups of the molecules maintaining an interaction with polar end groups of membrane constituents (e.g. phosphotidylcholine) while also spanning the intervening hydrophobic core. This has been shown to be membrane stabilizing, particularly in ischemia. Completely hydrocarbon carotenoids (e.g. lycopene, β-carotene) may float randomly in the hydrophobic core, and this has been demonstrated to be membrane destabilizing.

Lateral methyl groups have a small total effect on the λₘₐₓ of a polyenic carotenoid (5 nm or less). Therefore, a slight improvement in overall antioxidant potency may be achieved by structural modification (e.g., removal of lateral methyl groups, or changing their positions, either symmetrically or asymmetrically).

Of the various end groups on typical C40 and shorter carotenoids, the β-ring, having the structure:

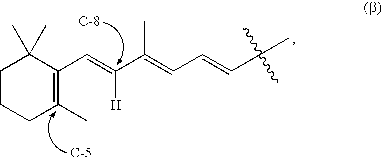

introduces steric hindrance (e.g., methyl groups at C-5 and hydrogen at C-8), preventing full co-planarity between the rings and the polyene chain. The resulting angle between the plane of the ring and the plane of the polyene chain is known as the torsion angle. Overall, orbital overlap (of the π electrons) is reduced, and therefore the contribution of the ring double bonds to the overall lambda max (λₘₐₓ) is relatively small.

In the absorbance spectrum, spectral fine structure is reduced, and the λₘₐₓ is at shorter wavelength than an acyclic carotenoid with the same number of double bonds. The β-ring double bond increases λₘₐₓ by approximately 5 nm. Ring-chain steric hindrance in this case may be reduced by removing one or both of the groups that contribute to the steric interaction (e.g., methyl at C-5, hydrogen at C-8). In a practical sense, movement of the methyl group is most facile. A general strategy of reducing ring-chain steric hindrance in cyclic carotenoids is highly beneficial towards achieving a greater absorbance in carotenoid analogs and derivatives.

In cyclic carotenoids, extending the conjugation throughout the ring may extend conjugation and increase λₘₐₓ concomitantly. Including an additional double bond in the 3,4 position of the β-ring, for example, may extend conjugation and increases λₘₐₓ by about 10 nm. Addition of a third double bond to the ring system may create the maximal situation in terms of conjugation, resulting in an aromatic phenyl ring, at least as regards six-membered cyclic rings.

In some embodiments, aromatic rings may be used as end groups for carotenoid compounds. The aryl phi (Φ) and chi (χ) rings are two such examples. The 1,2,5-trimethylphenyl end group (Φ), having the structure:

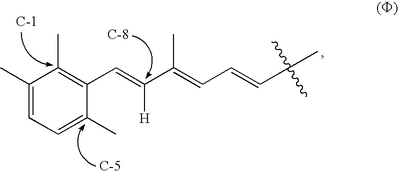

increases conjugation maximally throughout the ring system. However, steric hindrance remains between the C-1 and C-5 methyl groups and the C-8 hydrogen group, causing a decrease in co-planarity between the ring and the chain. The net effect is that the Φ-end group contribution to lambda max is approximately equal to that of the β-ring, having the structure:

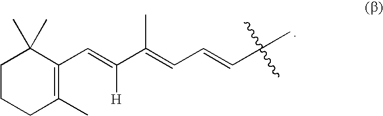

However, the 1,2,3-trimethylphenyl (χ) end group removes steric hindrance by the C-5 methyl group and the C-8 hydrogen. In the case of the naturally-occurring carotenoid renierapurpurin (χ,χ-carotene) having the structure:

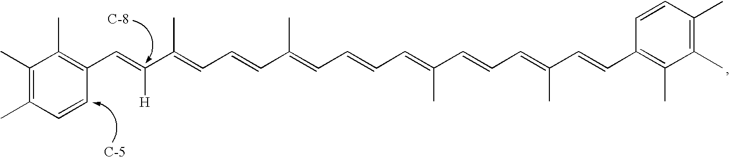

this extends the lambda max to that of the acyclic naturally-occurring carotenoid lycopene 2F. Modulation of steric hindrance in ring-chain systems may assist in increasing λₘₐₓ.

In some embodiments, ring contraction may be employed to relieve steric hindrance. For example, the 2-nor β-ring (a 5-membered ring) achieves a shift in λₘₐₓ of 15 to 20 nm for the cyclic pair zeaxanthin 2C and 2,2′dinor zeaxanthin having the structure:

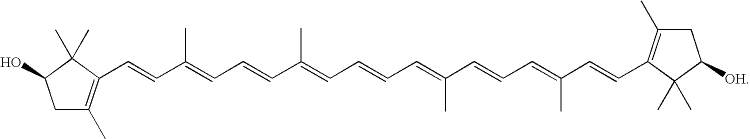

The same increase in λₘₐₓ is achieved in the cyclic pair astaxanthin 2E and actinioerythrin (actinioerythrol) having the structure:

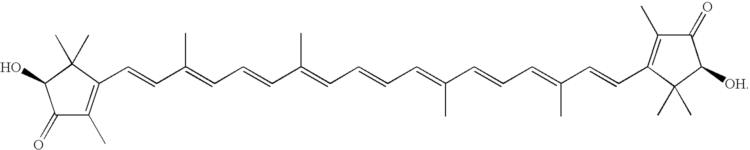

In some embodiments, ring contraction may be employed to relieve steric hindrance and increase the lambda max of carotenoid analogs and derivatives. Increasing lambda max may vastly improve the antioxidant potency while essentially conserving the absolute molecular length of a carotenoid.

Cyclic carotenoids, such as β-carotene:

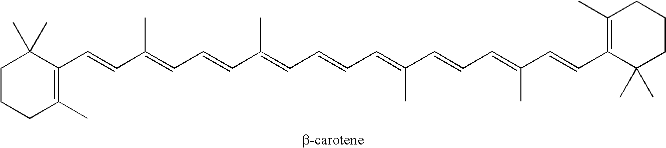

may contain five or six-membered headgroup rings that are geometrically staggered or out-of-plane with respect to the bridging conjugated polyene. Non-planarity between non-planar rings and the planar polyene serves to relieve or accommodate associated steric interactions, but lessens the orbital overlap of π-electrons of the headgroup ring double bonds with the polyene, in comparison to acyclic carotenoids having the same number of double bonds. As a result, non-planarity between headgroup rings and the polyene limits the energy-absorbing capacity. Reducing steric interactions between the headgroup rings and the polyene chain serves to enhance light and energy-absorbing capacity and/or antioxidant bioactivity of carotenoids and analogs.

Structural comparison of the related xanthophylls astaxanthin and actinioerythrol:

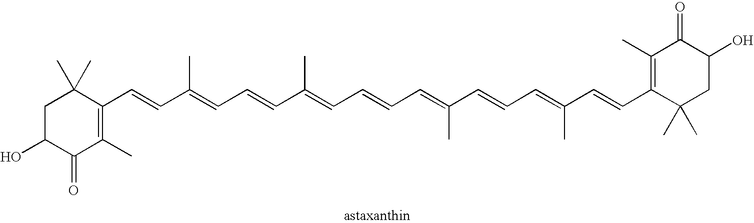

reveals that astaxanthin possesses six-membered headgroup rings, while actinioerythrol contains five-membered rings. Replacing six-membered headgroup rings with more planar five-membered rings serves to enhance energy-absorption capacity, in that a maximum electronic absorption (λₘₐₓ) difference of greater than 50 nm (478 nm for astaxanthin, 530 nm for actinioerythrol) is achieved.

Phenoxy chemical moieties can impart light and energy-absorption capacity, and/or antioxidant bioactivity, as exhibited by flavonoid-based natural pigments (cyanidin, delphinidin), and medicinally relevant polyphenols (resveratrol, tocopherols). Interestingly, some carotenoids, such as dihydroxyisorenieratene, possess enhanced phenoxy moieties, such that these functionalities are in-conjugation with the carotenoid polyene. Carotenoid headgroup rings exhibiting a significant degree of in-conjugation unsaturation, such as the carotenoids violerythrin and dihydroxyisorenieratene:

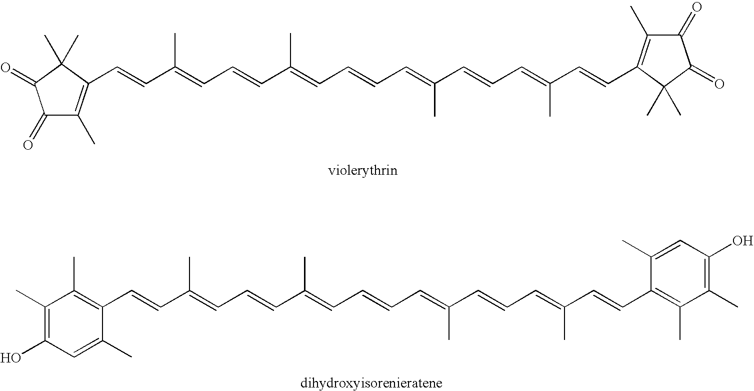

approach maximal conjugation (π-electron orbital overlap), and therefore possess enhanced energy-absorption capacity and/or antioxidant bioactivity.

In some embodiments, headgroup ring contraction may be employed to relieve steric hindrance and increase the light and energy-absorbing capacity and/or antioxidant bioactivity of carotenoids, carotenoid analogs, and intermediates:

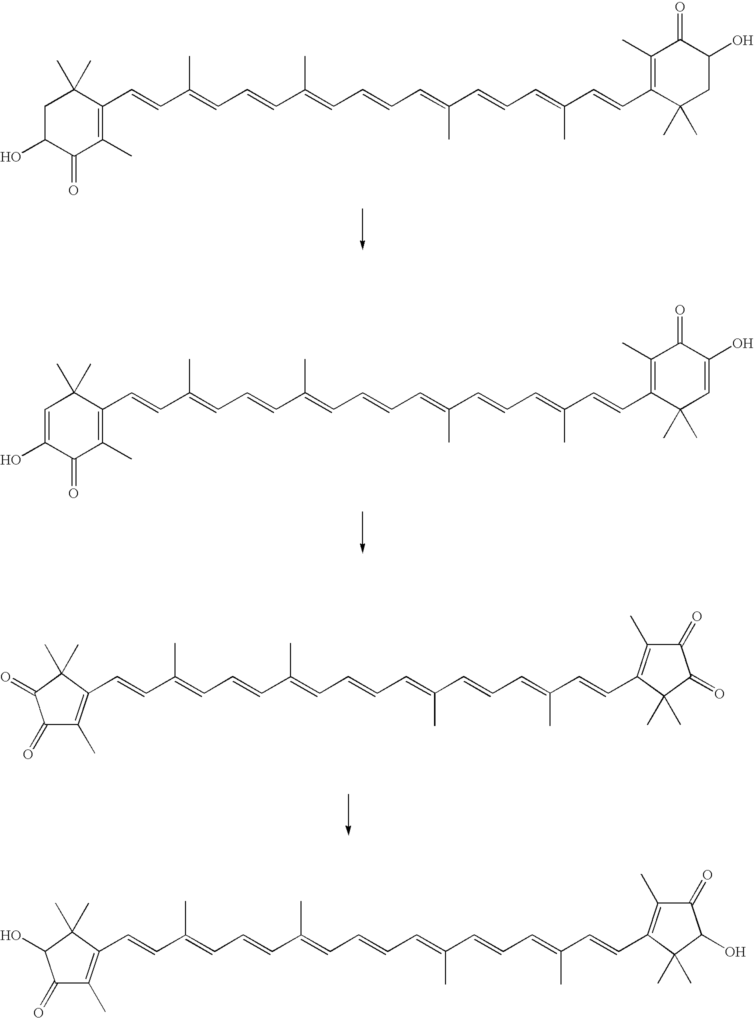

For example, astaxanthin may be selectively oxidized and its headgroup rings contracted to eventually yield a related xanthophyll (actinioerythrol) possessing enhanced energy-absorbing capacity and antioxidant characteristics.

In some embodiments, xanthophylls containing same or mixed aryl headgroup rings are synthesized, such that in-conjugation phenoxy moieties are incorporated into the core polyene chromophore, enhancing energy-absorbing capacity and antioxidant characteristics. Specifically, a prepared phenoxy-containing ylide may be coupled to crocetin dialdehyde to yield either a double-coupled product (containing same headgroup rings), or a single-coupled product (containing one headgroup ring and one unreacted aldehyde). The single-coupled product may be recycled and coupled to a prepared phenoxy-containing ylide to yield either same or mixed headgroup phenoxy carotenoids including, but not limited to:

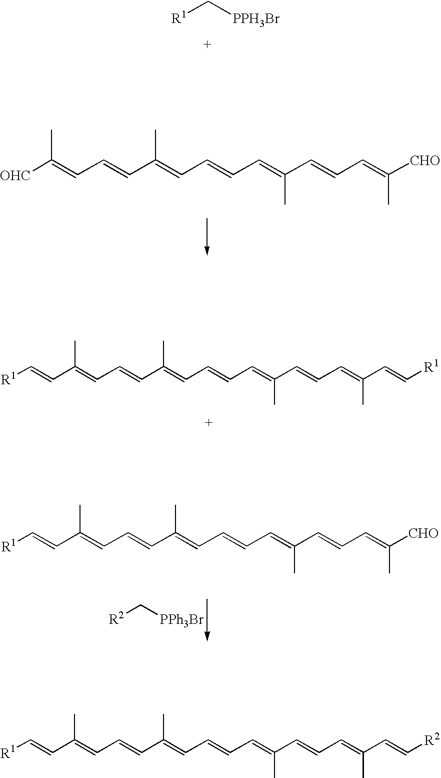

where R¹ and R² are independently:

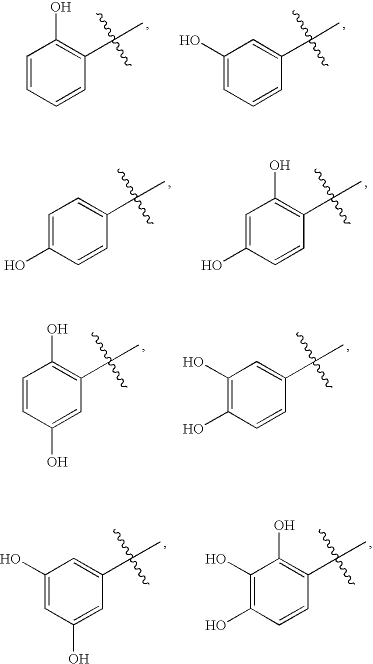

In some embodiments, water-solubility and/or water-dispersibility may be modulated by introduction of ester- and ether-linked moieties to ring and acyclic end groups. In some embodiments, introduction of additional synthetic handles on cyclic and acyclic carotenoids may be accomplished using retrometabolic drug design. For example, in the 1,2,3-trimethylphenyl (χ) end group, introduction of hydroxyl groups at the 1, 2, and 3 positions (or some subset thereof) may facilitate introduction of ester- and ether-linked moieties. Highly hydrophilic moieties (e.g. phosphates) and co-antioxidants (e.g. vitamin C, vitamin E, polyphenolics, flavonoids) may be joined directly or through the use of clinically relevant linkers to carotenoids. For carotenoids having a 1,2,3-trimethylphenyl (χ) end group, for example, a stoichiometric ratio of 6 hydrophilic- and/or co-antioxidant moieties to one polyene chain may be achieved. This has the desired therapeutic and clinical effect of increasing the ratio of co-antioxidant to carotenoid during administration, and increasing the water solubility and/or dispersibility of the novel synthetic compound. In addition, in neutral conditions, it has been shown that the phenolic hydroxyl groups in 1,2,5 and 1,2,3 aryl carotenoids have little effect on the absorbance spectrum (and hence λₘₐₓ); however, in basic conditions, ionization causes a substantial bathochromic shift. This may be particularly preferable in mammalian systems, where physiological pH is maintained in the slightly basic range (7.35-7.45).

In some embodiments, novel synthetic carotenoids with improved structural characteristics may be obtained by synthetic modification using one or more of the following principles:

removal of ring-chain steric hindrance (e.g., through ring contraction);

introduction and/or extension of ring conjugation; and

introduction of synthetic handles to increase solubility/dispersibility and stoichiometric ratios of ester/ether moieties.

The synthesis of certain carotenoids analogs and derivatives is presented herein. In some embodiments, methods and reactions described herein may be used to synthesize naturally-occurring carotenoids. Naturally-occurring carotenoids may include astaxanthin and actinionerythrol as well as other carotenoids. Some of the other carotenoids may include carotenoids such as, for example, zeaxanthin, carotenediol, nostoxanthin, crustaxanthin, canthaxanthin, isozeaxanthin, hydroxycanthaxanthin, tetrahydroxy-carotene-dione, lutein, and lycopene.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

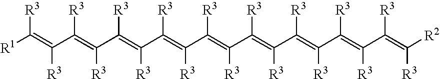

where each R³ is independently hydrogen or methyl, and where each R¹ and R² are independently:

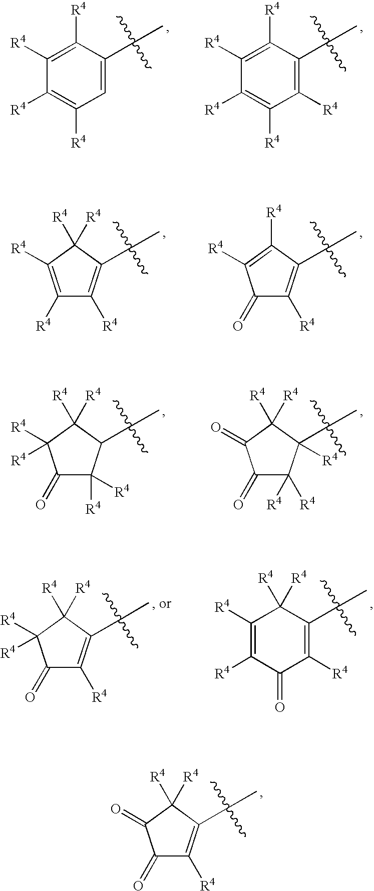

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(Re)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(R⁶)₃; —C(NR⁶)-aryl-N⁺(R⁶)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

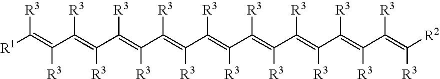

where each R³ is independently hydrogen or methyl, and where each R¹ and R² are independently:

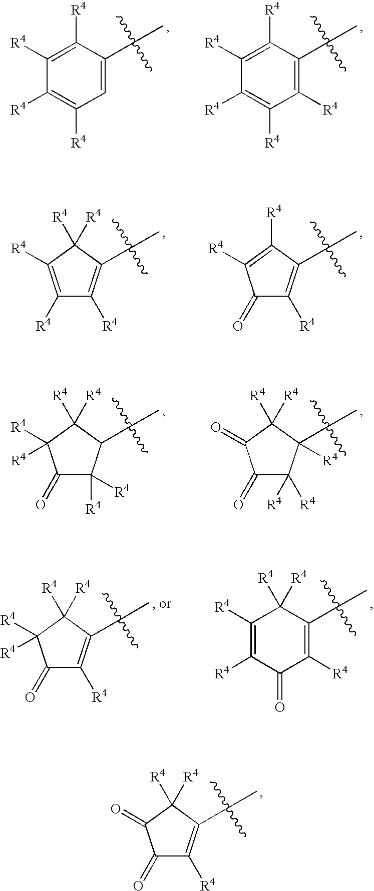

where each R⁴ is independently hydrogen, methyl, —OH, or —OR⁵ wherein at least two R⁴ groups are —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(Re)₃; —C(NR⁶)-aryl-N⁺(R⁶)₃; —C(NR⁶)-alkyl-CO₂R⁷; —C(R⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; and where n is 1 to 9.

In some embodiments, a chemical compound may have the structure:

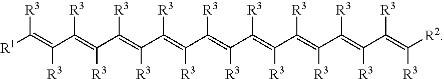

Each R³ may be independently hydrogen or methyl. Each R¹ and R² may be independently:

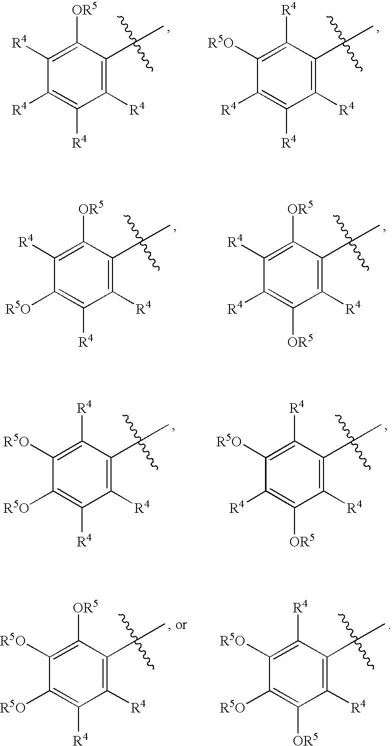

Each R⁴ may be independently hydrogen or methyl. Each R⁵ may be independently: hydrogen, alkyl; aryl; -alkyl-N(R⁶)₂; -aryl-N(R)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-allyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. In some embodiments, R⁵ is an amino acid, amino acid derivative, or amino acid analog. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. R⁹ may be a co-antioxidant. In some embodiments, n is 1 to 9. In some embodiments, the chemical compound may form at least a portion of a composition. In some embodiments, a method of inhibiting and/or ameliorating a disease associated with reactive oxygen species and/or other radical and non-radical species may comprise administering to a subject the chemical compound.

In some embodiments, a chemical compound having the structure:

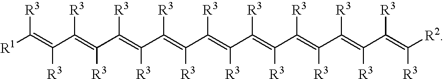

Each R³ may be independently hydrogen or methyl. Each R¹ and R² may be independently:

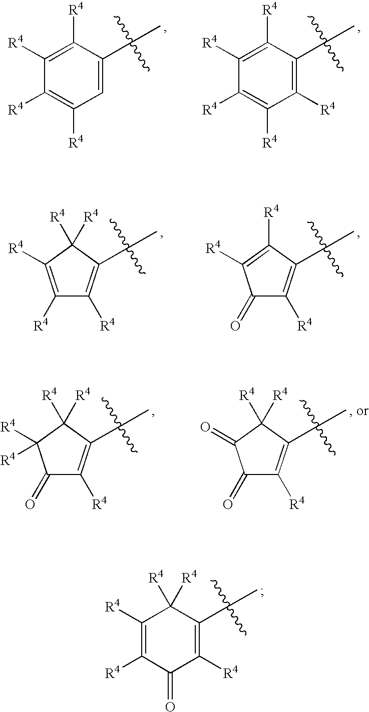

Each R⁴ may be independently hydrogen, methyl, —OH, or —OR⁵. At least one R⁴ group may be —OR⁵. Each R⁵ may be independently: hydrogen; alkyl; aryl; -alkyl-N(R⁶)₂; -aryl-N(R⁶)₂; -alkyl-CO₂H; -aryl-CO₂H; —C(O)—R⁷; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant. In some embodiments, at least two R⁴ groups may be —OR⁵. Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. At least one R⁵ may be -alkyl-N(R⁶)₂; -aryl-N(R⁶)₂; -alkyl-CO₂H; -aryl-CO₂H; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant. R⁶ may be hydrogen, alkyl, or aryl. R⁷ may be hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. In some embodiments, n is 1 to 9. In some embodiments, the chemical compound may form at least a portion of a composition. In some embodiments, the chemical compound may form at least a portion of a composition. In some embodiments, a method of inhibiting and/or ameliorating a disease associated with reactive oxygen species and/or other radical and non-radical species may comprise administering to a subject the chemical compound.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

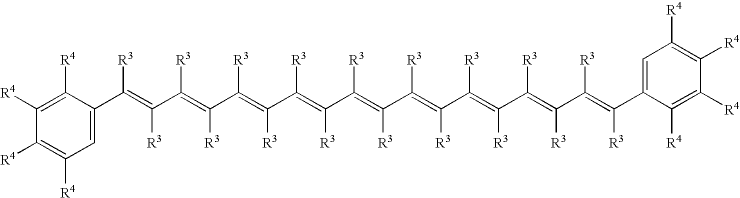

where each R³ is independently hydrogen or methyl, where each R⁴ is independently hydrogen, methyl, —OH, or —OR⁵ wherein at least two R⁴ groups are —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

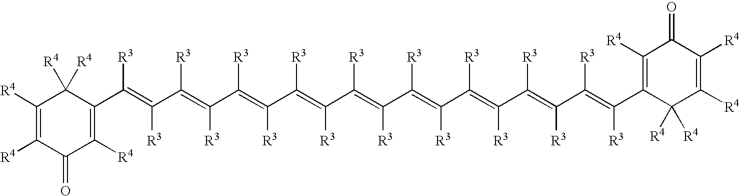

where each R³ is independently hydrogen or methyl, where each R⁴ is independently hydrogen, methyl, —OH, or —OR⁵ wherein at least one R⁴ groups are —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, allyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

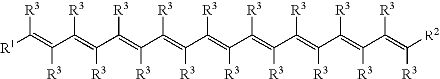

where each R³ is independently hydrogen or methyl, and where each R¹ and R² are independently:

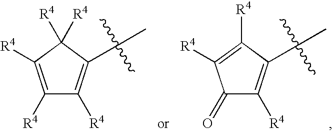

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

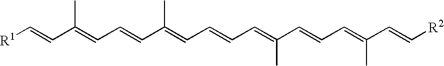

where each R¹ and R² are independently:

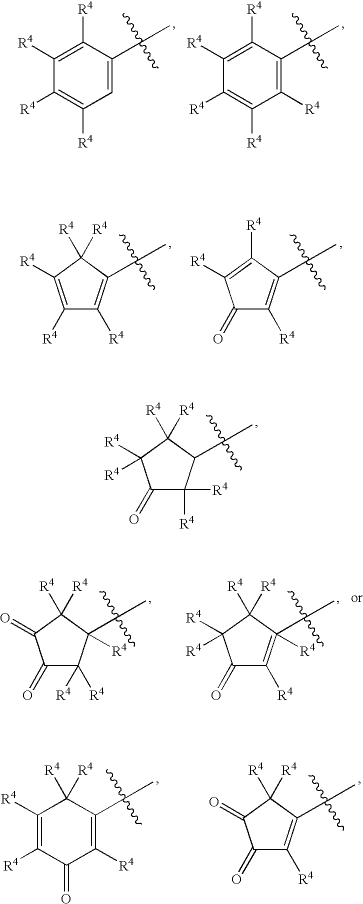

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

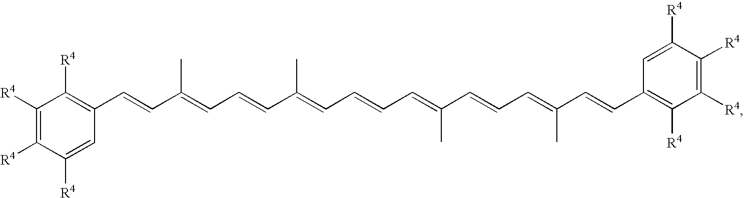

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

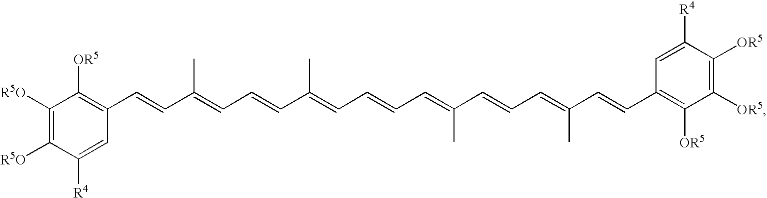

where each R⁴ is independently hydrogen, methyl, —OH, or —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-allyl-N(R⁶)₂; —C(O)-aryl-N(R⁶)₂; —C(O)-alkyl-N⁺(R)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R¹ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

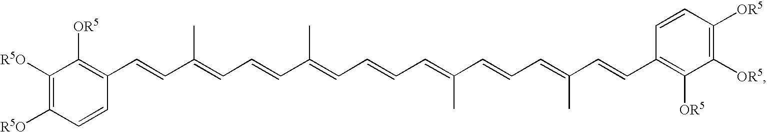

wherein each R⁵ is independently: hydrogen, alkyl; aryl; -alkyl-N(R⁶)₂; -aryl-N(Re)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

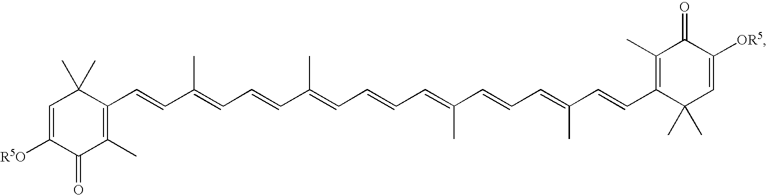

wherein each R⁵ is independently: hydrogen; hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

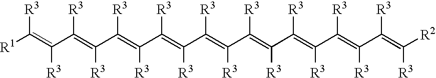

where each R³ is independently hydrogen or methyl, and where each R¹ and R² are independently:

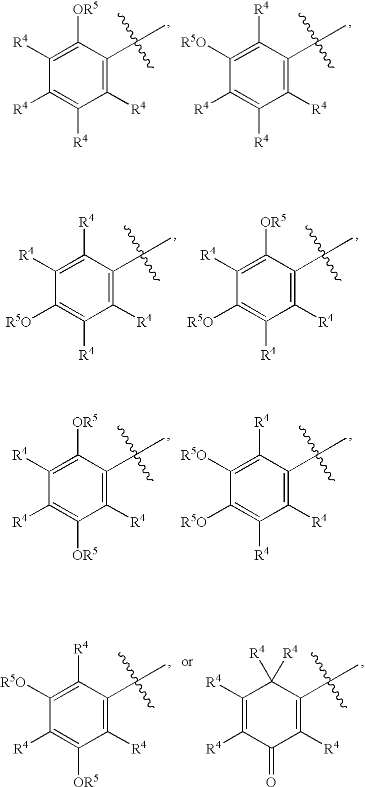

where each R⁴ is independently hydrogen, methyl, —OH, or —OR¹; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; and where n is 1 to 9.

In some embodiments, a composition may include one or more carotenoids, carotenoid analogs, carotenoid derivatives, and pharmaceutically acceptable derivatives of carotenoids, carotenoid analogs, and carotenoid derivatives having the general structure:

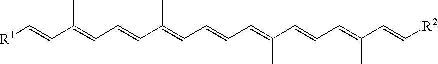

where each R¹ and R² are independently:

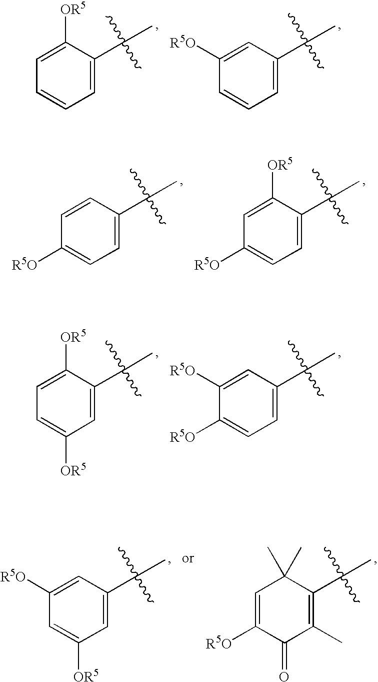

Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(Re)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may include hydrogen, alkyl, or aryl. R⁷ may include hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may range from 1 to 9.

In some embodiments, a substituent —OR⁵ may include

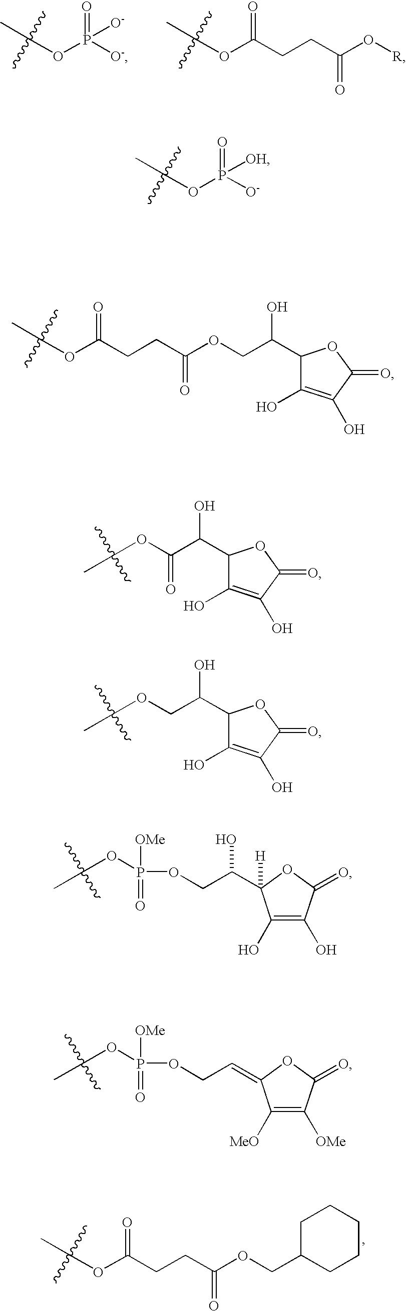

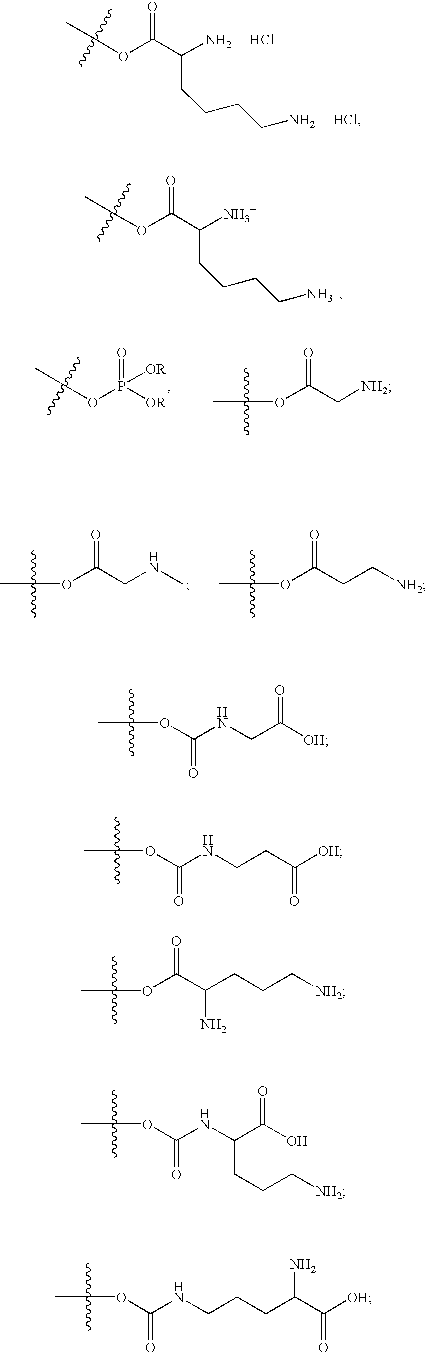

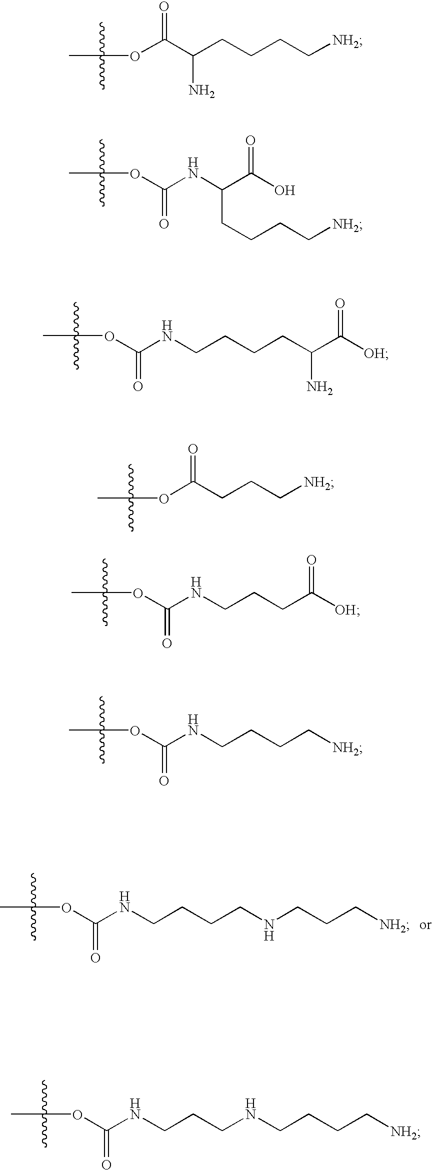

wherein each R is independently H, alkyl, aryl, benzyl, Group IA metal, or co-antioxidant.

Some specific embodiments of —OR⁵ may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, polyphenolics, flavonoids, flavonoid analogs, or flavonoid derivatives), or combinations thereof of derivatives or analogs of carotenoids. Flavonoids may include, for example, quercetin, xanthohumol, isoxanthohumol, or genistein. Polyphenolics may include, for example, resveratrol.

Vitamin E may generally be divided into two categories including tocopherols having a general structure

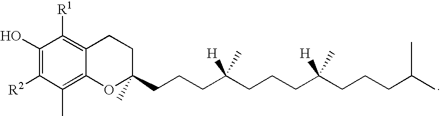

Alpha-tocopherol is used to designate when R¹=R²═CH₃. Beta-tocopherol is used to designate when R¹═CH₃ and R²═H. Gamma-tocopherol is used to designate when R¹═H and R²═CH₃. Delta-tocopherol is used to designate when R¹=R²═H.

The second category of Vitamin E may include tocotrienols having a general structure

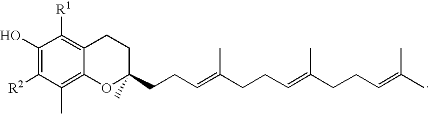

Alpha-tocotrienol is used to designate when R¹=R²═CH₃. Beta-tocotrienol is used to designate when R¹═CH₃ and R²═H. Gamma-tocotrienol is used to designate when R¹═H and R²═CH₃. Delta-tocotrienol is used to designate when R¹=R²═H.

Quercetin, a flavonoid, has the structure

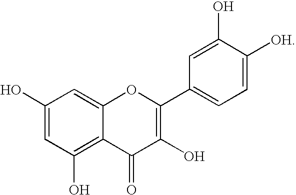

In some embodiments, one or more co-antioxidants may be coupled to a carotenoid or carotenoid derivative or analog. Derivatives of one or more carotenoid analogs may be formed by coupling one or more free hydroxy groups of the co-antioxidant to a portion of the carotenoid.

When R⁵ is an amino acid derivative or a peptide, coupling of the amino acid or the peptide is accomplished through an ester linkage or a carbamate linkage. Specifically, an ester linked amino acid group —OR⁵ has the general structures:

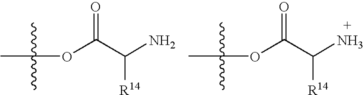

Depending on if the free form of the salt form is desired. A carbamate linked amino acid group —OR⁵ will have the general structure:

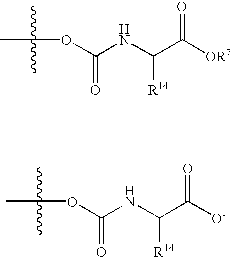

Depending on if the free form of the salt form is desired. For both ester linked and carbamate linked amino acids, the group R¹⁴ represents an amino acid side chain. Specifically, R¹⁴ can be: —H (glycine); —CH₃ (alanine); —CH(CH₃)—CH₃ (valine); —CH₂—CH(CH₃)—CH₃ (leucine); —CH(CH₃)—CH₂—CH₃ (isoleucine); —CH₂-Ph (phenylalanine); —CH₂—CH₂—S—CH₃ (methionine); —CH₂—OH (serine); —CH(CH₃)—OH (threonine); —CH₂—SH (cysteine); —CH₂-Ph-OH (tyrosine); —CH₂—C(O)—NH₂ (aspargine); —CH₂—CH₂-C(O)—NH₂ (glutamine); —CH₂—CO₂H (aspartic acid); —CH₂—CH₂-CO₂H (glutamic acid); —CH₂—CH₂-CH₂—CH₂—NH₂ (lysine); —CH₂—CH₂-CH₂—NH₂ (ornithine); —CH₂—CH₂—CH₂—NH—C(NH)—NH₂ (arginine);

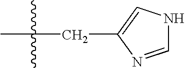

(histidine); and

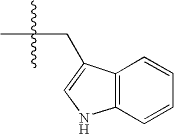

(tryptophan). Amino acid side chains can be in the neutral form (as depicted above) or in a salt form. When R¹⁴ represents the side chain from the amino acid proline, the following compounds result:

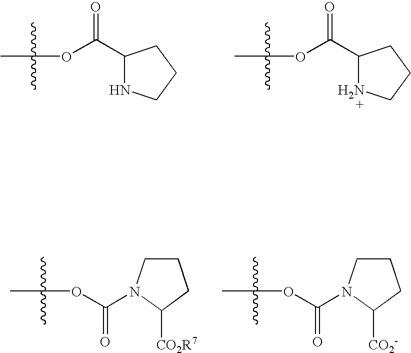

When R⁸ is an amino acid derivative or a peptide, coupling of the amino acid or the peptide is accomplished through an amide linkage. The amide linkage may be formed between the terminal carboxylic acid group of the linker attached to the xanthophyll carotene and the amine of the amino acid or peptide.

When R⁵ is a carbohydrate, R⁵ includes, but is not limited to the following side chains: —CH₂—(CHOH)ₙ—CO₂H; —CH₂—(CHOH)ₙ—CHO; —CH₂—(CHOH)ₙ—CH₂OH; —CH₂—(CHOH)ₙ—C(O)—CH₂OH;

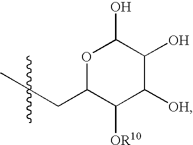

where R¹⁰ is hydrogen or

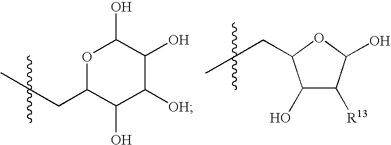

where R¹³ is hydrogen or —OH.

When R⁵ is a nucleoside, R⁵ may have the structure:

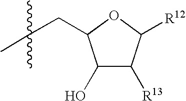

where R¹² is a purine or pyrimidine base, and R¹³ is hydrogen or —OH.

When R⁵ is —C(O)—[C₆-C₂₄ saturated hydrocarbon], the substituent, R⁵, is derived from coupling of a saturated fatty acid with the carotenoid parent structure. Examples of saturated fatty acids include, but are not limited to: hexanoic acid (caproic acid); octanoic acid (caprylic acid); decanoic acid (capric acid); dodecanoic acid (lauric acid); tridecanoic acid; tetradecanoic acid (myristic acid); pentadecanoic acid; hexadecanoic acid (palmitic acid); heptadecanoic acid (margaric acid); octadecanoic acid (stearic acid); eicosanoic acid (arachidic acid); docosanoic acid (behenic acid); tricosanoic acid; and tetracosanoic acid (lignoceric acid).

When R⁵ is —C(O)—[C₆-C₂₄ monounsaturated hydrocarbon], the substituent, R⁵, is derived from coupling of a monounsaturated fatty acid with the carotenoid parent structure. Examples of monounsaturated fatty acids include, but are not limited to: 9-tetradecenoic acid (myristoleic acid); 9-hexadecenoic acid (palmitoleic acid); 11-octadecenoic acid (vaccenic acid); 9-octadenoic acid (oleic acid); 11-eicosenoic acid; 13-docosenoic acid (erucic acid); 15-tetracosanoic acid (nervonic acid); 9-trans-hexadecenoic acid (palmitelaidic acid); 9-trans-octadecenoic acid (elaidic acid); 8-eicosaenoic acid; and 5-eicosaenoic acid.

When R⁵ is —C(O)—[C₆-C₂₄ polyunsaturated hydrocarbon], the substituent, R⁵, is derived from coupling of a polyunsaturated fatty acid with the carotenoid parent structure. Examples of polyunsaturated fatty acids include, but are not limited to omega-3 polyunsaturated fatty acids, omega-6 polyunsaturated fatty acids; and conjugated polyunsaturated fatty acids. Examples of omega-3 polyunsaturated fatty acids include, but are not limited to: 9,12,15-octadecatrienoic acid (alpha-linolenic acid); 6,9,12,15-octadecatetraenoic acid (stearidonic acid); 11,14,17-eicosatrienoic acid (eicosatrienoic acid (ETA)); 8,11,14,17-eicsoatetraenoic acid (eicsoatetraenoic acid); 5,8,11,14,17-eicosapentaenoic acid (eicosapentaenoic acid (EPA)); 7,10,13,16,19-docosapentaenoic acid (docosapentaenoic acid (DPA)); 4,7,10,13,16,19-docosahexaenoic acid (docosahexaenoic acid (DHA)); 6,9,12,15,18,21-tetracosahexaenoic acid (nisinic acid); 9E,11Z,15E-octadeca-9,11,15-trienoic acid (rumelenic acid); 9E,11Z,13Z,15E-octadeca-9,11,13,15-trienoic acid (α-parinaric acid); and all trans-octadeca-9,11,13,15-trienoic acid (β-parinaric acid). Examples of omega-6 polyunsaturated fatty acids include, but are not limited to: 9,12-octadecadienoic acid (linoleic acid); 6,9,12-octadecatrienoic acid (gamma-linolenic acid); 11,14-eicosadienoic acid (eicosadienoic acid); 8,11,14-eicosatrienoic acid (homo-gamma-linolenic acid); 5,8,11,14-eicosatetraenoic acid (arachidonic acid); 13,16-docosadienoic acid (docosadienoic acid); 7,10,13,16-docosatetraenoic acid (adrenic acid); 4,7,10,13,16-docosapentaenoic acid (docosapentaenoic acid); 8E,10E,12Z-octadecatrienoic acid (calendic acid); 10E,12Z-octadeca-9,11-dienoic acid; 8E,10E,12Z-octadecatrienoic acid (α-calendic acid); 8E,10E,12E-octadecatrienoic acid (β-calendic acid); 8E,10Z,12E-octadecatrienoic acid (jacaric acid); and 5Z,8Z,10E,12E,14Z-eicosanoic acid (bosseopentaenoic acid). Examples of conjugated polyunsaturated fatty acids include, but are not limited to: 9Z,11E-octadeca-9,11-dienoic acid (rumenic acid); 10E,12Z-octadeca-9,11-dienoic acid; 8E,10E,12Z-octadecatrienoic acid (α-calendic acid); 8E,10E,12E-octadecatrienoic acid (β-calendic acid); 8E,10Z,12E-octadecatrienoic acid (jacaric acid); 9E,11E,13Z-octadeca-9,11,13-trienoic acid (α-eleostearic acid); 9E,11E,13E-octadeca-9,11,13-trienoic acid (3-eleostearic acid); 9Z,11Z,13E-octadeca-9,11,13-trienoic acid (catalpic acid); 9E,11Z,13E-octadeca-9,11,13-trienoic acid (punicic acid); 9E,11Z,15E-octadeca-9,11,15-trienoic acid (rumelenic acid); 9E,11Z,13Z,15E-octadeca-9,11,13,15-trienoic acid (α-parinaric acid); all trans-octadeca-9,11,13,15-trienoic acid (β-parinaric acid); and 5Z,8Z,10E,12E,14Z-eicosanoic acid (bosseopentaenoic acid).

Derivatives or analogs may be derived from any known carotenoid (naturally or synthetically derived). Specific examples of naturally occurring carotenoids which compounds described herein may be derived from include for example actinioerytlrol, capsorubin, renierapurpurin, isorenieratene, violerytlrin, astacene, zeaxanthin, lutein, lycophyll, astaxanthin, and lycopene.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

“Water-soluble” structural carotenoid analogs or derivatives are those analogs or derivatives that may be formulated in aqueous solution, either alone or with one or more excipients. Water-soluble carotenoid analogs or derivatives may include those compounds and synthetic derivatives that form molecular self-assemblies, and may be more properly termed “water dispersible” carotenoid analogs or derivatives. Water-soluble and/or “water-dispersible” carotenoid analogs or derivatives may be preferred in some embodiments.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/ml -10 mg/mL. In certain embodiments, water-soluble carotenoid analogs or derivatives may have a water solubility of greater than about 20 mg/mL. In certain embodiments, water-soluble carotenoid analogs or derivatives may have a water solubility of greater than about 25 mg/mL. In some embodiments, water-soluble carotenoid analogs or derivatives may have a water solubility of greater than about 50 mg/mL.

In some embodiments, a composition may include a carotenoid analog or carotenoid derivative having the structure (I):

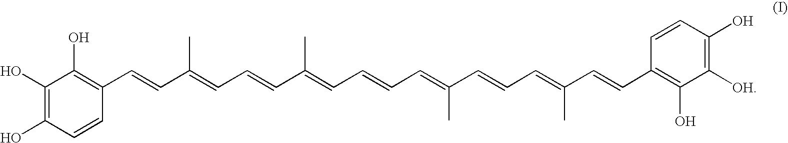

In some embodiments, a composition may include a carotenoid analog or carotenoid derivative having the structure (II):

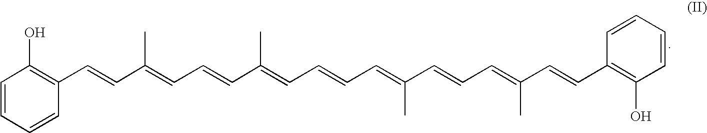

In some embodiments, a composition may include a carotenoid analog or carotenoid derivative having the structure (III):

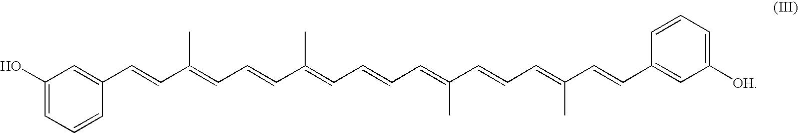

In some embodiments, a composition may include a carotenoid analog or carotenoid derivative having the structure (IV):

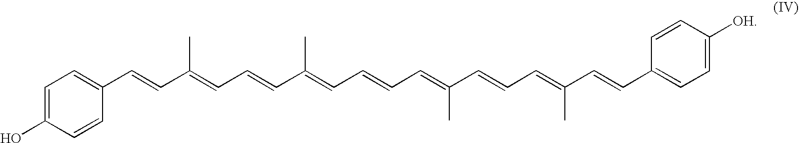

In some embodiments, a composition may include a carotenoid analog or carotenoid derivative having the structure (V):

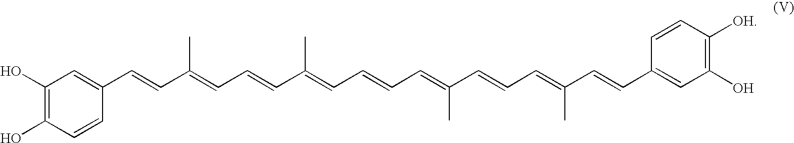

In some embodiments, a composition may include a carotenoid analog or carotenoid derivative having the structure (VI):

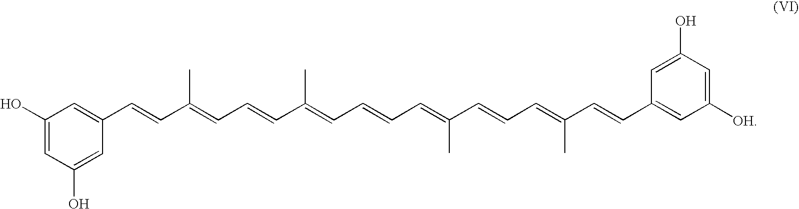

In some embodiments, a composition may include a carotenoid analog or carotenoid derivative having the structure (VII):

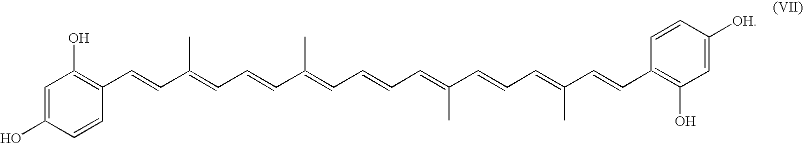

In some embodiments, a composition may include a carotenoid analog or carotenoid derivative having the structure (VIII):

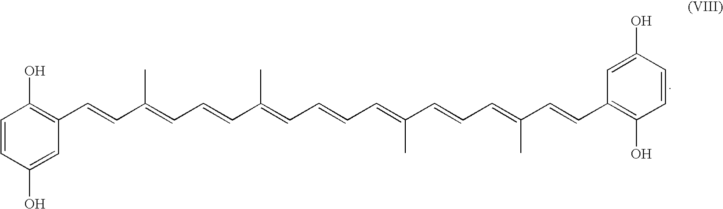

Such a compound may be used as an intermediate to synthesize other carotenoid analogs or carotenoid derivatives.

In some embodiments, carotenoid analogs and derivatives may be synthesized using the general process shown in Scheme I below.

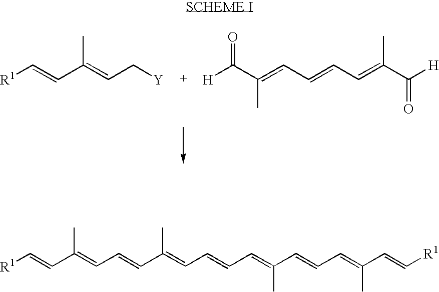

Where each R¹ is independently:

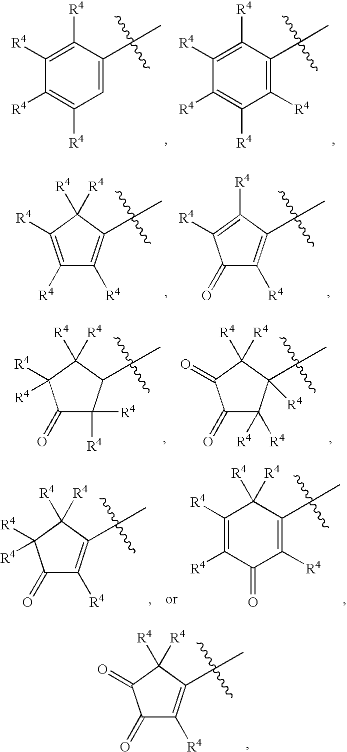

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R³ is hydrogen; alkyl; aryl; —P(O)(OR⁷)₂; —S(O)(OR⁷)₂; an amino acid; a peptide, a carbohydrate; a nucleoside, or a co-antioxidant; where n is 1 to 9; where Y is PR⁶₃, SO₂R⁶, or M⁺; and M is Li, Na, or MgBr.

Coupling of two “head units” with the C₁₀-aldehyde yields carotenoid. Coupling may be accomplished using a Wittig coupling (Y is PR⁶₃), sulphone coupling (Y is SO₂R⁶), or condensation reaction (Y is M⁺). The C₁₀ aldehyde is commercially available. Described herein are various methods of synthesizing the appropriate headpiece. The following U.S. patents, all of which are incorporated herein by reference, describe the synthesis of various carotene and carotenoid synthesis intermediates: U.S. Pat. Nos. 4,245,109 to Mayer et al., 4,283,559 to Broger et al, 4,585,885 to Bernhard et al., 4,952,716 to Lukac et al., and 6,747,177 to Ernst et al.

In some embodiments, carotenoid analogs and derivatives may be synthesized using the general process shown in Scheme II below.

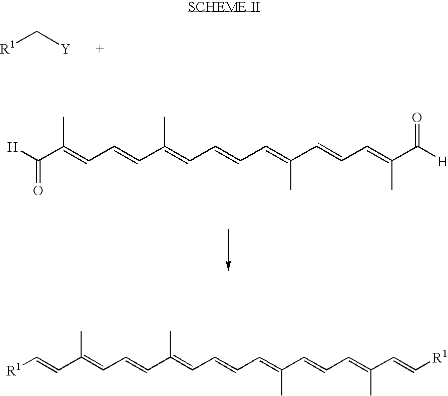

Where each R¹ is independently:

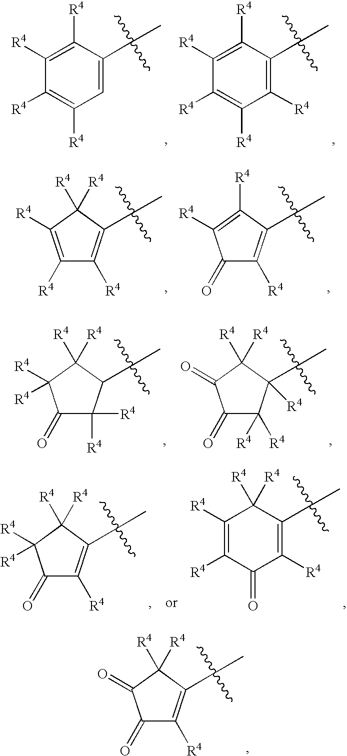

where each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; where R⁶ is hydrogen, alkyl, or aryl; wherein R⁷ is hydrogen, alkyl, aryl, benzyl, Group IA metal or a co-antioxidant; where R⁸ is hydrogen; alkyl; aryl; —P(O)(OR⁷)₂; —S(O)(OR⁷)₂; an amino acid; a peptide, a carbohydrate; a nucleoside, or a co-antioxidant; where n is 1 to 9; where Y is PR⁶₃, SO₂R⁶, or M; and M is Li, Na, or MgBr.

In some embodiments, a carotenoid chemical intermediate (i.e., as depicted in Scheme II) may include a compound having the general structure:

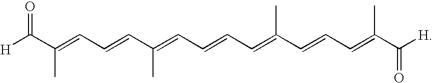

In some embodiments, carotenoid chemical intermediates may be used to synthesize naturally occurring carotenoids as well as carotenoid analogs and carotenoid derivatives. Carotenoid chemical intermediates may be used to synthesize naturally occurring carotenoids such as lycopene and lycopene analogs and lycopene derivatives.

In some embodiments, a synthetic sequence which may be used to make the chemical intermediate pictured above is depicted in Scheme III.

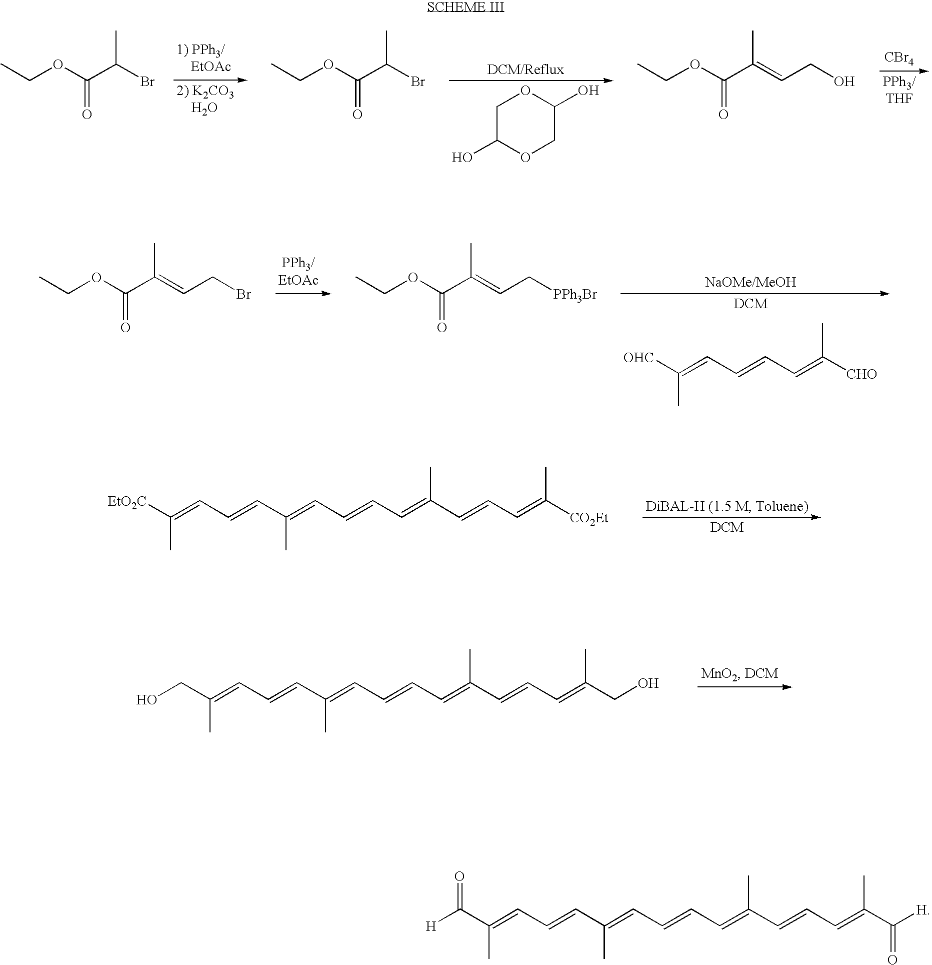

In some embodiments, carotenoid analogs and derivatives (e.g., compound (I)) may be synthesized using the general process depicted in Scheme IV below.

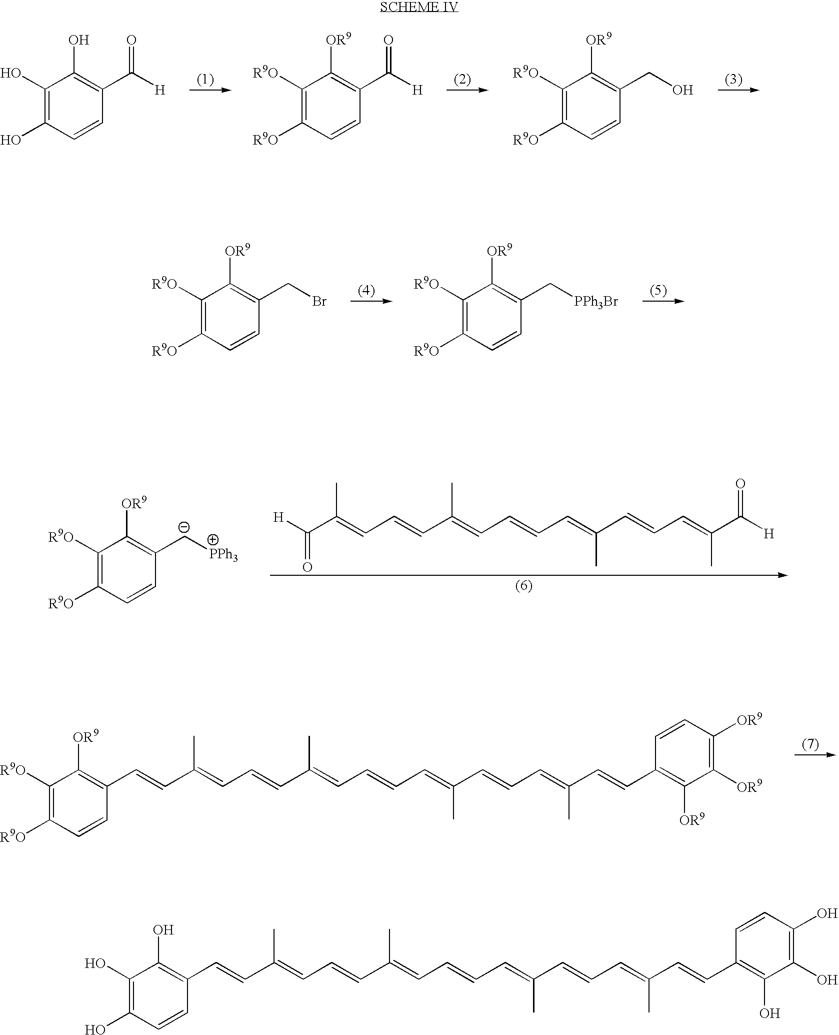

R⁹ may include any appropriate protecting group known to one skilled in the art. R⁹ may include, but is not limited to, alkyl, aryl, or silyl. For example reaction (1) may include protecting any hydroxy groups with a known protecting group (e.g., triethylsilane (TES)). Reaction (2) may include reducing the aldehyde to an alcohol. Reduction of the aldehyde to an alcohol may be accomplished via a hydride source (e.g., sodium borohydride). Reaction (3) may include halogenation of the formed alcohol. Any halogen may be substituted for the alcohol (e.g., Br, Cl, or I). There are many methods of halogenation known to one skilled in the art, but should be chosen based of course on the resiliency of the chosen protecting group (e.g., R⁹). Reaction (4) may include transformation of the halogen into a triaryl phosphorous derivative (e.g., with triphenyl phosphine (Ph₃P)). Reaction (5) may include formation of an intermediate zwitter ion. In this instance a zwitter ion may be formed using a base capable of abstracting a hydrogen forming the zwitter ion. Reaction (6) may a coupling reaction (e.g., a Wittig reaction), which couples one or more of the zwitter ions with an aldehyde (e.g., dialdehyde). Upon formation of the carotenoid intermediate, any protecting groups (e.g., R⁹) may be removed during reaction (7) using a reagent appropriate to the protecting group (e.g., pyridinium p-toluenesulfonate (PPTS)). In some embodiments, compound (I) may be used as an intermediate for making other carotenoid analogs and carotenoid derivatives described herein.

In some embodiments, a carotenoid, carotenoid analog, and/or carotenoid derivative having the general structure:

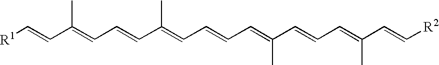

where each R¹ and R² are independently:

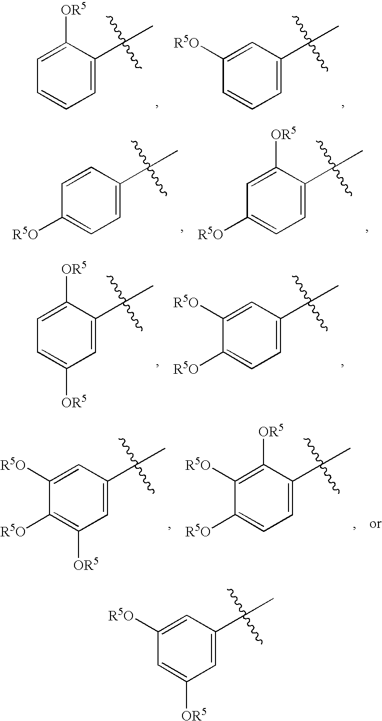

may be synthesized using the general process depicted in Scheme IVa (details of specific embodiments are discussed in the Examples). Each R⁵ may be independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant. R⁶ may include hydrogen, alkyl, or aryl. R⁷ may include hydrogen, alkyl, aryl, benzyl, Group IA metal or a co-antioxidant. R⁸ may be hydrogen; alkyl; aryl; —P(O)(OR⁷)₂; —S(O)(OR⁷)₂; an amino acid; a peptide, a carbohydrate; a nucleoside, or a co-antioxidant. n may range from 1 to 9.

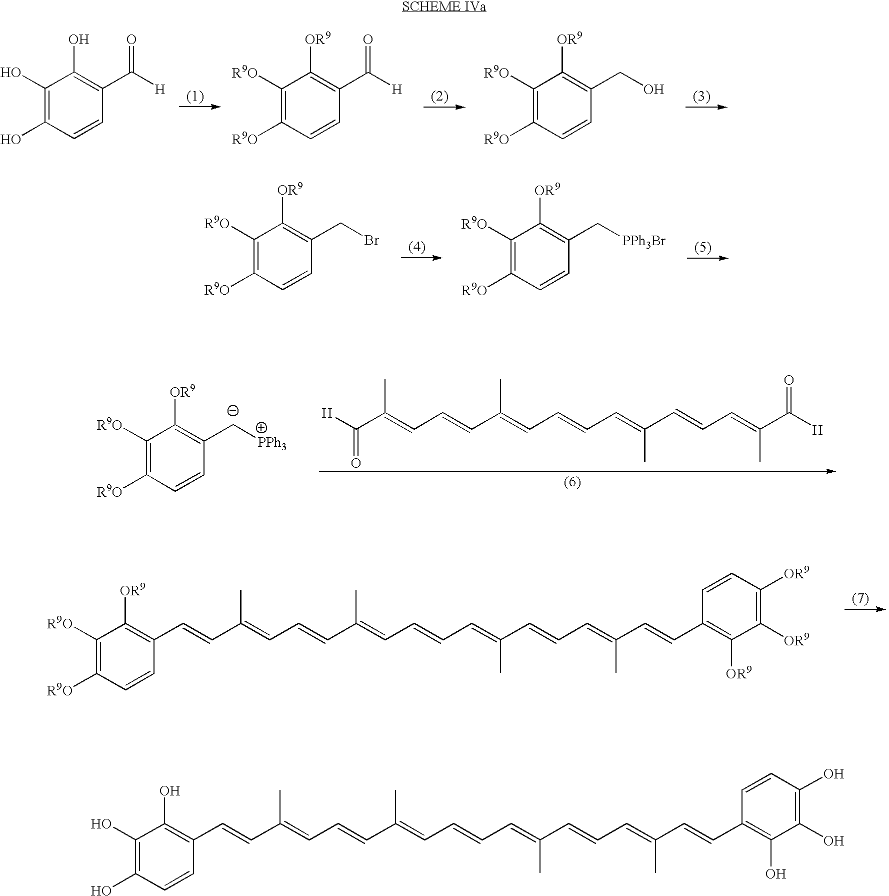

R⁹ may include any appropriate protecting group known to one skilled in the art. R⁹ may include, but is not limited to, alkyl, aryl, or silyl. For example reaction (1) may include protecting any hydroxy groups with a known protecting group (e.g., triethylsilane (TES)). Reaction (2) may include reducing the aldehyde to an alcohol. Reduction of the aldehyde to an alcohol may be accomplished via a hydride source (e.g., sodium borohydride). Reaction (3) may include halogenation of the formed alcohol. Any halogen may be substituted for the alcohol (e.g., Br, Cl, or I). There are many methods of halogenation known to one skilled in the art, but should be chosen based of course on the resiliency of the chosen protecting group (e.g., R⁹). Reaction (4) may include transformation of the halogen into a triaryl phosphorous derivative (e.g., with triphenyl phosphine (Ph₃P)). Reaction (5) may include formation of an intermediate zwitter ion. In this instance a zwitter ion may be formed using a base capable of abstracting a hydrogen forming the zwitter ion. Reaction (6) may a coupling reaction (e.g., a Wittig reaction), which couples one or more of the zwitter ions with an aldehyde (e.g., dialdehyde). Upon formation of the carotenoid intermediate, any protecting groups (e.g., R⁹) may be removed during reaction (7) using a reagent appropriate to the protecting group (e.g., pyridinium p-toluenesulfonate (PPTS)). In some embodiments, compound (I) may be used as an intermediate for making other carotenoid analogs and carotenoid derivatives described herein.

In some embodiments, carotenoid analogs and derivatives (e.g., compound (I)) may be synthesized using the process depicted in Scheme V below.

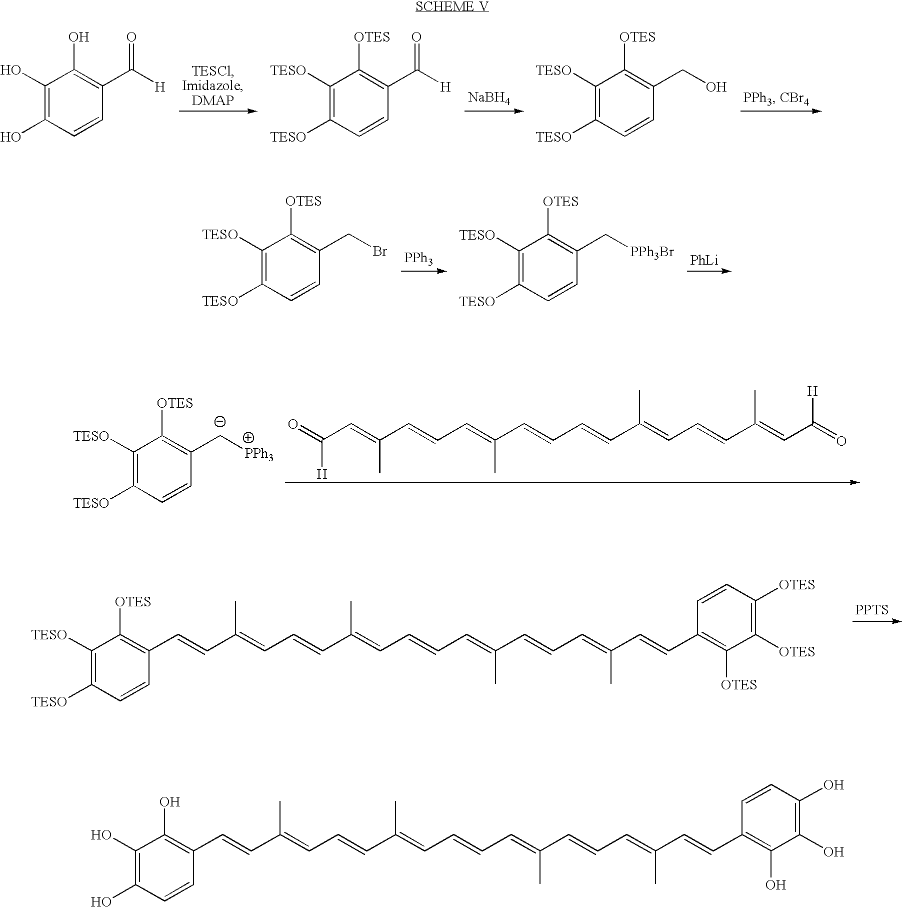

In some embodiments, carotenoid analogs and derivatives (e.g., compound (I)) may be synthesized using the process depicted in Scheme VI below.

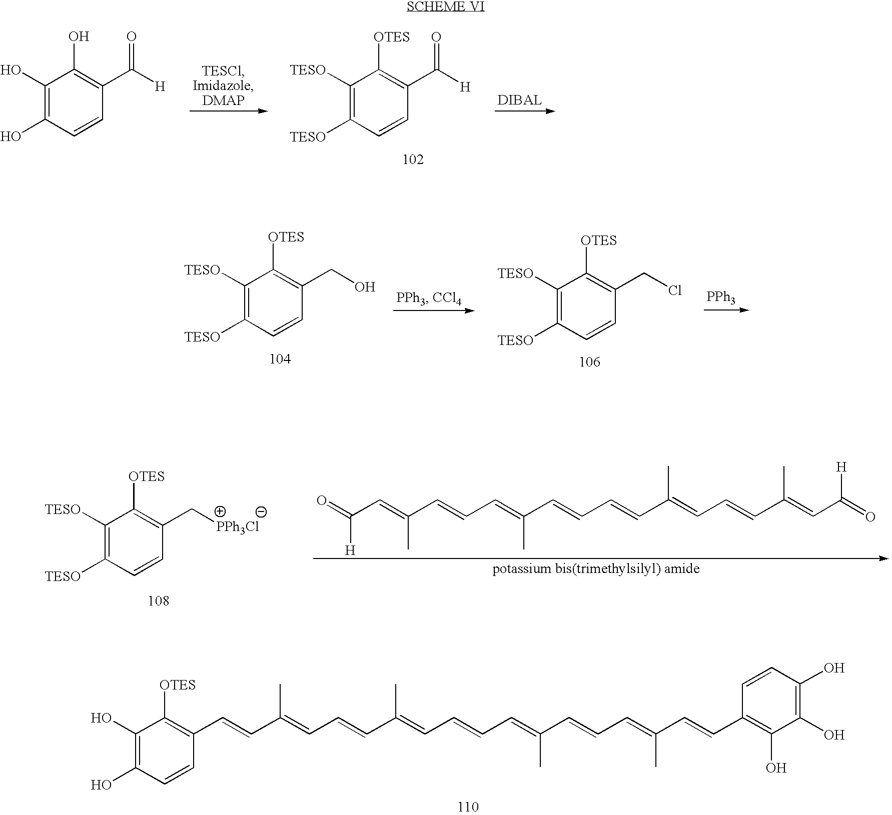

Experimentals for the above numbered compounds may be found herein below.

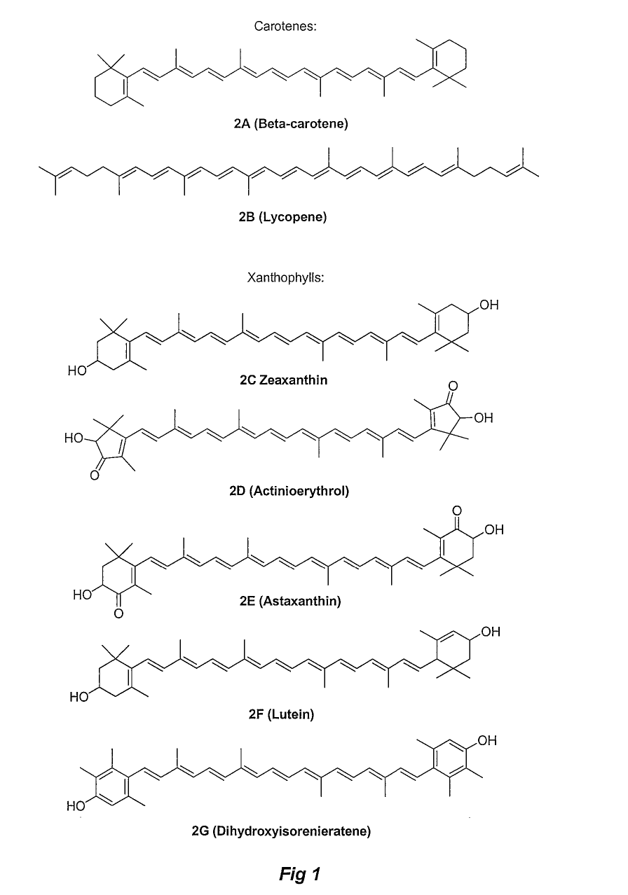

In an embodiment, carotenoid derivatives may be synthesized from naturally-occurring carotenoids. The carotenoids may include structures 2A-2G depicted in FIG. 1. In some embodiments, the carotenoid derivatives may be synthesized from a naturally-occurring carotenoid including one or more alcohol substituents. In other embodiments, the carotenoid derivatives may be synthesized from a derivative of a naturally-occurring carotenoid including one or more alcohol substituents. The synthesis may result in a single stereoisomer. The synthesis may result in a single geometric isomer of the carotenoid derivative. The synthesis/synthetic sequence may include any prior purification or isolation steps carried out on the parent carotenoid. Synthesis of carotenoid derivatives and analogs can be found in U.S. Published Patent Application Nos. 2004-0162329 and 2005-0113372, both of which are incorporated herein by reference.

In some embodiments, the administration of carotenoids, carotenoid analogs, carotenoid derivatives, or pharmaceutically acceptable derivatives of carotenoids, carotenoid analogs, and carotenoid derivatives may inhibit and/or ameliorate the occurrence of diseases in subjects. Diseases that may be treated include any disease that involves production of reactive oxygen species and/or other radical and non-radical species (for example singlet oxygen, a reactive oxygen species but not a radical). In some embodiments, carotenoids, carotenoid analogs, carotenoid derivatives, or pharmaceutically acceptable derivatives of carotenoids, carotenoid analogs, and carotenoid derivatives may be used to treat a disease that involves production of reactive oxygen species. Oxidation of DNA, proteins, and lipids by reactive oxygen species and other radical and non-radical species has been implicated in a host of human diseases. Radicals may make the body more susceptible to other disease-initiating factors, may inhibit endogenous defenses and repair processes, and/or may enhance the progression of incipient disease(s). The administration of carotenoids, carotenoid analogs, carotenoid derivatives, or pharmaceutically acceptable derivatives of carotenoids, carotenoid analogs, and carotenoid derivatives by one skilled in the art—including consideration of the pharmacokinetics and pharmacodynamics of therapeutic drug delivery—is expected to inhibit and/or ameliorate said disease conditions. In the first category are those disease conditions in which a single organ is primarily affected, and for which evidence exists that radicals and/or non-radicals are involved in the pathology of the disease. The following are diseases that may be inhibited and/or ameliorated by the administration of carotenoids, carotenoid analogs, carotenoid derivatives, or pharmaceutically acceptable derivatives of carotenoids, carotenoid analogs, and carotenoid derivatives. These examples are not to be seen as limiting, and additional disease conditions will be obvious to those skilled in the art.
Head, Eyes, Ears, Nose, and Throat: age-related macular degeneration (ARMD), retinal detachment, hypertensive retinal disease, uveitis, choroiditis, vitreitis, ocular hemorrhage, degenerative retinal damage, cataractogenesis and cataracts, retinopathy of prematurity, Meuniere's disease, drug-induced ototoxicity (including aminoglycoside and furosemide toxicity), infectious and idiopathic otitis, otitis media, infectious and allergic sinusitis, head and neck cancer;
Central Nervous System (brain and spinal cord): senile dementia (including Alzheimer's dementia), Neuman-Pick's disease, neurotoxin reactions, hyperbaric oxygen effects, Parkinson's disease, cerebral and spinal cord trauma, hypertensive cerebrovascular injury, stroke (thromboembolic, thrombotic, and hemorrhagic), infectious encephalitis and meningitis, allergic encephalomyelitis and other demyelinating diseases, amyotrophic lateral sclerosis (ALS), multiple sclerosis, neuronal ceroid lipofuscinoses, ataxia-telangiectasia syndrome, aluminum, iron, and other heavy metal(s) overload, primary brain carcinoma/malignancy and brain metastases;
Cardiovascular: arteriosclerosis, atherosclerosis, peripheral vascular disease, myocardial infarction, chronic stable angina, unstable angina, idiopathic surgical injury (during CABG, PTCA), inflammatory heart disease [as measured and influenced by C-reactive protein (CRP) and myeloperoxidase (MPO)], vascular restenosis, low-density lipoprotein oxidation (ox-LDL), cardiomyopathies, cardiac arrhythmia (ischemic and post-myocardial infarction induced), congestive heart failure (CHF), drug toxicity (including adriamycin and doxorubicin), Keshan disease (selenium deficiency), trypanosomiasis, alcohol cardiomyopathy, venous stasis and injury (including deep venous thrombosis or DVT), thrombophlebitis;
Pulmonary: asthma, reactive airways disease, chronic obstructive pulmonary disease (COPD or emphysema), hyperoxia, hyperbaric oxygen effects, cigarette smoke inhalation effects, environmental oxidant pollutant effects, acute respiratory distress syndrome (ARDS), bronchopulmonary dysplasia, mineral dust pneumoconiosis, adriamycin toxicity, bleomycin toxicity, paraquat and other pesticide toxicities, chemical pneumonitis, idiopathic pulmonary interstitial fibrosis, infectious pneumonia (including fungal), sarcoidosis, asbestosis, lung cancer (small- and large-cell), anthrax infection, anthrax toxin exposure;
Renal: hypertensive renal disease, end-stage renal disease, diabetic renal disease, infectious glomerulonephritis, nephrotic syndrome, allergic glomerulonephritis, type I-IV hypersensitivity reactions, renal allograft rejection, nephritic antiglomerular basement membrane disease, heavy metal nephrotoxicity, drug-induced (including aminoglycoside, furosemide, and non-steroidal anti-inflammatory) nephrotoxicity, rhabdomyolisis, renal carcinoma;
Hepatic: carbon tetrachloride liver injury, endotoxin and lipopolysaccharide liver injury, chronic viral infection (including Hepatitis infection), infectious hepatitis (non-viral etiology), hemachromatosis, Wilson's disease, acetaminophen overdose, congestive heart failure with hepatic congestion, cirrhosis (including alcoholic, viral, and idiopathic etiologies), hepatocellular carcinoma, hepatic metastases;
Gastrointestinal: inflammatory bowel disease (including Crohn's disease, ulcerative colitis, and irritable bowel syndrome), colon carcinoma, polyposis, infectious diverticulitis, toxic megacolon, gastritis (including Helicobacter pylori infection), gastric carcinoma, esophagitis (including Barrett's esophagus), gastro-esophageal reflux disease (GERD), Whipple's disease, gallstone disease, pancreatitis, abetalipoproteinemia, infectious gastroenteritis, dysentery, nonsteroidal anti-inflammatory drug-induced toxicity;
Hematopoietic/Hematologic: Pb (lead) poisoning, drug-induced bone marrow suppression, protoporphyrin photo-oxidation, lymphoma, leukemia, porphyria(s), parasitic infection (including malaria), sickle cell anemia, thallasemia, favism, pernicious anemia, Fanconi's anemia, post-infectious anemia, idiopathic thrombocytopenic purpura (ITP), autoimmune deficiency syndrome (AIDS);
Genitourinary: infectious prostatitis, prostate carcinoma, benign prostatic hypertrophy (BPH), urethritis, orchitis, testicular torsion, cervicitis, cervical carcinoma, ovarian carcinoma, uterine carcinoma, vaginitis, vaginismus;
Musculoskeletal: osteoarthritis, rheumatoid arthritis, tendonitis, muscular dystrophy, degenerative disc disease, degenerative joint disease, exercise-induced skeletal muscle injury, carpal tunnel syndrome, Guillan-Barre syndrome, Paget's disease of bone, ankylosing spondilitis, heterotopic bone formation; and
Integumentary: solar radiation injury (including sunburn), thermal injury, chemical and contact dermatitis (including Rhus dermatitis), psoriasis, Bloom syndrome, leukoplakia (particularly oral), infectious dermatitis, Kaposi's sarcoma.

In the second category are multiple-organ conditions whose pathology has been linked convincingly in some way to radical and non-radical injury: aging, including age-related immune deficiency and premature aging disorders, cancer, cardiovascular disease, cerebrovascular disease, radiation injury, alcohol-mediated damage (including Wemicke-Korsakoff's syndrome), ischemia-reperfusion damage, inflammatory and auto-immune disease, drug toxicity, amyloid disease, overload syndromes (iron, copper, etc.), multi-system organ failure, and endotoxemia/sepsis.

Maladies, which may be treated with carotenoids, carotenoid analogs, carotenoid derivatives, or pharmaceutically acceptable derivatives of carotenoids, carotenoid analogs, and carotenoid derivatives, may include, but are not limited to, cardiovascular inflammation, hepatitis C infection, cancer (hepatocellular carcinoma and prostate), macular degeneration, rheumatoid arthritis, stroke, Alzheimer's disease, and/or osteoarthritis. In an embodiment, the administration of carotenoids, carotenoid analogs, carotenoid derivatives, or pharmaceutically acceptable derivatives of carotenoids, carotenoid analogs, and carotenoid derivatives to a subject may inhibit and/or ameliorate the occurrence of ischemia-reperfusion injury in subjects. In some embodiments, carotenoids, carotenoid analogs, carotenoid derivatives, or pharmaceutically acceptable derivatives of carotenoids, carotenoid analogs, and carotenoid derivatives may be administered to a subject alone or in combination with other carotenoid analogs or derivatives. The occurrence of ischemia-reperfusion injury in a human subject that is experiencing, or has experienced, or is predisposed to experience myocardial infarction, stroke, peripheral vascular disease, venous or arterial occlusion and/or restenosis, organ transplantation, coronary artery bypass graft surgery, percutaneous transluminal coronary angioplasty, and cardiovascular arrest and/or death may be inhibited or ameliorated by the administration of therapeutic amounts of carotenoids, carotenoid analogs, carotenoid derivatives, or pharmaceutically acceptable derivatives of carotenoids, carotenoid analogs, and carotenoid derivatives to the subject.

### EXAMPLES

Having now described the invention, the same will be more readily understood through reference to the following example(s), which are provided by way of illustration, and are not intended to be limiting of the present invention.

Regarding the synthesis and characterization of compounds described herein, reagents were purchased from commercial sources and used as received unless otherwise indicated. Solvents for reactions and isolations were reagent grade and used without purification unless otherwise indicated. All of the following reactions were performed under nitrogen (N₂) atmosphere. LC/MS was recorded on an Agilent 1100 LC/MSD VL system; column: Zorbax Eclipse XDB-C18 Rapid Resolution (4.6×75 mm, 3.5 μm); temperature: 25° C.; starting pressure: 128 bar, flow rate: 1.0 mL/min; mobile phase (A=0.025% TFA in H₂O, B=0.025% TFA in acetonitrile); PDA Detector: 470 nm and 214 nm. Gradient program: 70% A/30% B (start), step gradient to 50% B over 5 min, step gradient to 98% B over 3.3 min, hold at 98% B over 16.9 min or longer.

### Example 1

### Preparation of 102

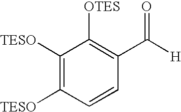

To a solution of chlorotriethylsilane (4 mL, 23.3 mmol) in N,N-dimethylformamide (20 mL) at room temperature, was added imidazole (1.56 g, 23.32 mmol). The reaction was stirred for 15 min, then 2,3,4-trihydroxybenzaldehyde (1.0 g, 6.48 mmol) and N,N-dimethylaminopyridine (712 mg, 5.84 mmol) were added. The reaction was stirred at room temperature under nitrogen atmosphere for 12 hr, at which time the solution was diluted with diethyl ether, quenched with aqueous NH₄Cl, and stirred for 5 min. The organic layer was washed with aqueous NH₄Cl, brine, and water. The combined organic layers were then dried over MgSO₄, filtered and concentrated to yield crude TES-protected phenolic benzaldehyde. R_{f} (hexane/diethyl ether 7:3)=0.88. MS (APCI): 10.574 min (>40% Area Under Curve), m/e: 497 (M+1) (25%), 498 (10%)

### Example 2

### Preparation of 104

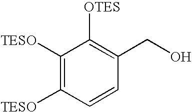

A solution of TES-protected 2,3,4-trihydroxybenzaldehyde (2.15 g, 4.32 mmol) in dichloromethane was cooled down to −78° C. and added dropwise a solution of diisobutylaluminum hydride (DIBAL) (1.0 M in dichloromethane, 8.64 mmol). The reaction mixture was stirred at −78° C. for 1 h, at which time the suspension was quenched with water (6 ml), warmed up to room temperature and solid NaHCO₃ and Et₂OAc (46 ml) were added to the mixture. This mixture was stirred at room temperature for 30 min, dried over MgSO₄, filtered and concentrated down to yield 2 g crude material. MS (APCI): 18.902 min (>58% Area Under Curve), m/e: 498 (M) (40%), 499 (20%),500 (10%).

### Example 3

### Preparation of 106

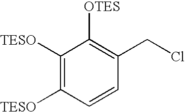

To a solution of crude TES-protected phenolic benzylic alcohol (1.0 g, 1.99 mmol) in CCl₄ (7 mL) was added PPh₃ (2.0 g, 7.63 mmol). The solution was stirred for 15 min at room temperature then refluxed for 2 hr at which time all solvents were removed. The residue was then re-suspended in hexane and filtered two times. After removing the solvents, crude TES-protected phenolic benzylic chloride was afforded. R_{f} (hexane/diethyl ether 9:1)=0.89.

### Example 4

### Preparation of 108

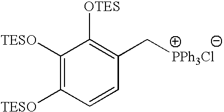

To a solution of TES-protected phenolic benzylic chloride (1.99 mmol) in benzene (10 mL) was added PPh₃ (5.2 g, 19.9 mmol). The solution was stirred for 15 min at room temperature under N₂, then refluxed for 12 hr at which time the solution was concentrated down to yield crude TES-protected phenolic benzylic triphenylphosphonium salt.

### Example 5

### Preparation of 110

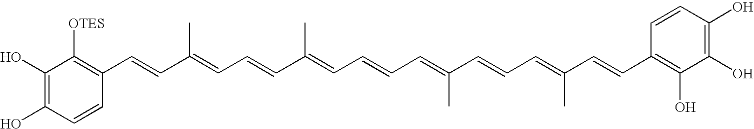

To a suspension of the TES-protected phenolic benzylic triphenylphosphonium salt (0.633 mmol) in tetrahydrofuran (4 mL) at 0° C. was added dropwise under nitrogen atmosphere potassium bis(trimethylsilyl)amide (1.25 mL, 0.633 mmol, 0.5 M in toluene). Crocetin dialdehyde (19 mg, 0.063 mmol) was then added, the solution was warmed to room temperature, and stirred for 12 hr. The solution was diluted with CH₂Cl₂ and the organic layer washed with aqueous NH₄Cl, brine, and water. The organic phase was dried over MgSO₄, the solution was filtered and concentrated down. After purification on a silica flash column with hexane/CH₂Cl₂ (4:1), and 1% triethylamine as the eluent system, four fractions were collected. Fraction 1 (dialdehyde, mono-triphenolic headgroup carotenoid): R_{f} (hexane/CH₂Cl₂ 2:3)=0.46 (yellow), 0.36 (orange); LC/MS (APCI): 9.30 min (7.71%), λₘₐₓ466 nm (100%), 445 nm (92%), 265 nm (18%), m/e: 295 (M+) (33%), 294 (100%), 240 (30%); 9.77 min (92.29%), λₘₐₓ484 nm (91%), 460 nm (100%), 279 nm (10%), m/e: 418 (M+) (38%), 417 (100%), 372 (10%); Fraction 2 (mono-triphenolic headgroup carotenoid, di-triphenolic headgroup carotenoid): R_{f} (hexane/CH₂Cl₂ 2:3)=0.36 (orange), 0.28 (red); LC/MS (APCI): 9.57 min (10.91%), λₘₐₓ484 nm (89%), 460 nm (100%), 279 nm (12%), m/e: 418 (M+) (34%), 417 (100%), 372 (11%); 10.01 min (89.08%), λₘₐₓ500 nm (80%), 469 nm (100%), 295 nm (18%); Fraction 3 (di-triphenolic headgroup carotenoid, mono-TES protected di-triphenolic headgroup carotenoid): R_{f} (hexane/CH₂Cl₂ 2:3)=0.28 (red), 0.18 (purple) LC/MS (APCI): 10.03 min (23.05%), λₘₐₓ500 nm (82%), 469 nm (100%), 294 nm (20%); 13.58 min (76.95%), λₘₐₓ502 nm (92%), 467 nm (100%), m/e: 654 (M+) (18%), 653 (20%), 606 (70%), 276 (66%), 240 (100%).

### Example 6

### Preparation of 1

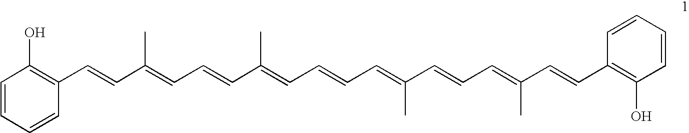

To a suspension of commercially available 2-(hydroxybenzyl)triphenylphosphonium bromide (40 mg, 0.0890 mmol) in tetrahydrofuran (5 ml) at 0° C., was added drop wise an ethereal solution of phenyllithium (20%, 0.936 mmol). The reaction was stirred for 10 min, then added dichloromethane (100 μL) to quench excess base and further added dropwise a concentrated solution of C20 crocetindialdehyde (13 mg, 0.0446 mmol) in dichloromethane (Scheme 1). The reaction was stirred at 0° C. for 10 min, warmed up to room temperature, and stirred for 30 min at which time the solution was diluted with Et₂O, quenched with aqueous NH₄Cl and stirred for 5 minutes. The organic layer was washed with aqueous NH₄Cl, brine, and water. The combined organic layers were then dried over MgSO₄, filtered, and concentrated to yield crude 2-hydroxy phenolic carotenoid (1). Purification on a silica flash column with hexane/tetrahydrofuran (6:4), 1% triethylamine as eluent system, gave target compound in 42% yield (51 mg): LC/MS (APCI): 10.482 min (86% Area Under Curve), λₘₐₓ472 nm (100%), 504 nm (85%), 448 nm (79%), m/e: 476 (M) (50%), 477 (M+1) (100%), 478 (40%).

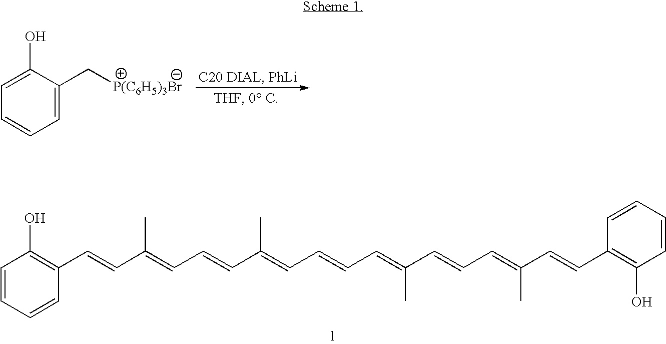

### Example 7

### Preparation of 2

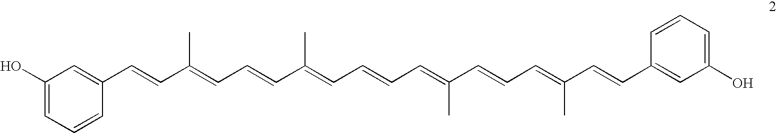

To a suspension of commercially available 3-hydroxybenzyl alcohol (100 mg, 0.806 mmol) in acetonitrile (4 ml) was added PPh₃-HBr (276 mg, 0.806 mmol) (Scheme 2). The reaction was refluxed for 12 h, after which the solution was cooled down to 0° C. The product that precipitated out was filtered, washed with ice-cold acetonotrile, and dried to afford 221 mg (61% yield) 3-(hydroxybenzyl)triphenylphosphonium bromide. MS (APCI): 3.525 min (>95% Area Under Curve), m/e: 369 (M-Br) (100%), 279 (P(O)(C₆H₅)₃) (30%), 263 (P(C₆H₅)₃) (20%). ¹H NMR (300 MHz, d₆-DMSO): δ: 8.69 ppm (1H, s), 7.09-6.78 ppm (15H, m), 6.17 ppm (1H, t, J=7.75 Hz), 5.87-5.84 ppm (1H, d, J=8.04 Hz), 5.58 (1H, s), 5.54-5.52 ppm (1H, d, J=7.47 Hz), 4.25-4.20 ppm (2H, d, J=15.68 Hz).

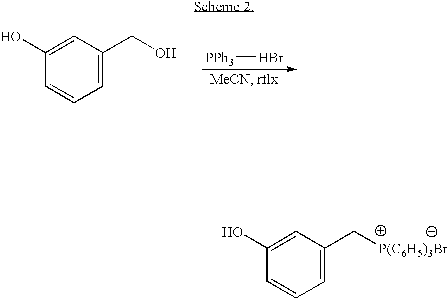

To a suspension of 3-(hydroxybenzyl)triphenylphosphonium bromide (50 mg, 0.110 mmol) in tetrahydrofuran (5 ml) at 0° C., was added drop wise an ethereal solution of phenyllithium (20%, 1.11 mmol). The reaction was stirred for 10 min, then added dichloromethane (100 μl) to quench excess base and further added drop wise a concentrated solution of C20 crocetindialdehyde (11 mg, 0.0371 mmol) in dichloromethane (Scheme 3). The reaction was stirred at 0° C. for 10 min and warmed up to room temperature and stirred for 30 min, at which time the solution was diluted with Et₂O, quenched with aqueous NH₄Cl and stirred for 5 minutes. The organic layer was washed with aqueous NH₄Cl, brine, and water. The combined organic layers were then dried over MgSO₄, filtered, and concentrated to yield crude 2-hydroxy phenolic carotenoid (2). LC/MS (APCI): 10.270 min (38% Area Under Curve), λₘₐₓ472 nm (100%), 504 nm (85%), 448 nm (79%), m/e: 476 (M) (90%), 477 (M+1) (30%), 478 (5%), 10.535 min (35% Area Under Curve), λₘₐₓ472 nm (100%), 504 nm (85%), 448 nm (79%), m/e: 476 (M) (100%), 477 (M+1) (40%), 478 (10%). Total Area Under Curve of the two peaks showed 73% yield conversion from C20 crocetindialdehyde.

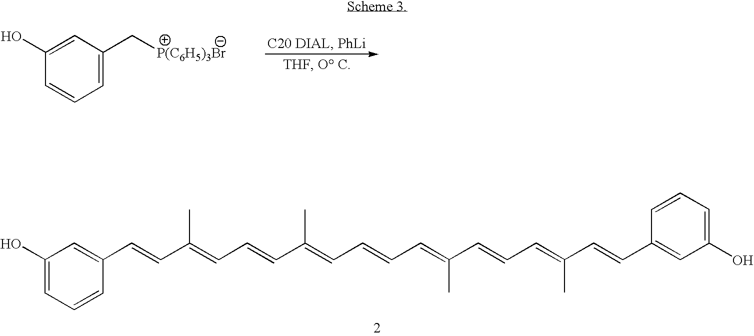

### Example 8

### Preparation of 3

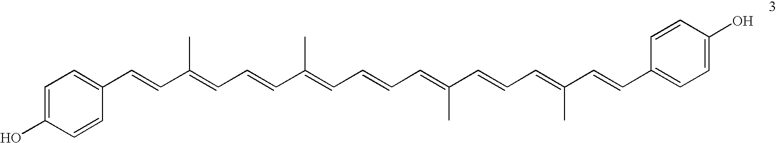

To a suspension of commercially available 4-hydroxybenzyl alcohol (100 mg, 0.806 mmol) in acetonitrile (4 ml) was added PPh₃-HBr (276 mg, 0.806 mmol) (Scheme 4). The reaction was refluxed for 12 h, after which the solution was cooled down to 0° C. The product that precipitated out was filtered, washed with ice-cold acetonotrile, and dried to yield 276 mg (76%) 4-(hydroxybenzyl)triphenylphosphonium bromide. MS (APCI): 3.452 min (>95% Area Under Curve), m/e: 369 (M-Br) (10%), 279 (P(O)(C₆H₅)₃) (20%), 263 (P(C₆H₅)₃) (100%). ¹H NMR (300 MHz, d₆-DMSO): δ: 8.79 ppm (1H, s), 7.15-6.81 ppm (15H, m), 5.93 ppm (2H, t, J=7.49 Hz), 5.77-5.75 ppm (2H, d, J=7.01 Hz), 4.21-4.16 (2H, d, J=14.57 Hz).

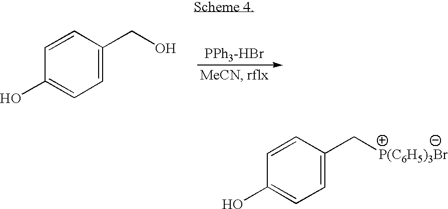

To a suspension of 4-(hydroxybenzyl)triphenylphosphonium bromide (50 mg, 0.111 mmol) in tetrahydrofuran (5 ml) at 0° C., was added drop wise an ethereal solution of phenyllithium (20%, 1.11 mmol). The reaction was stirred for 10 min, then added dichloromethane (100 μl) to quench excess base and further added drop wise a concentrated solution of C20 crocetindialdehyde (11 mg, 0.0371 mmol) in dichloromethane (Scheme 5). The reaction was stirred at 0° C. for 10 min and warmed up to room temperature and stirred for 30 min, at which time the solution was diluted with Et₂O, quenched with aqueous NH₄Cl and stirred for 5 minutes. The organic layer was washed with aqueous NH₄Cl, brine, and water. The combined organic layers were then dried over MgSO₄, filtered and concentrated down to yield crude 3-hydroxy phenolic carotenoid (3). LC/MS (APCI): 10.196-10.642 min (64% Area Under Curve), λₘₐₓ472 nm (100%), 504 nm (85%), 448 mm (79%), m/e: 476 (M) (40%), 477 (M+1) (70%), 478 (20%).

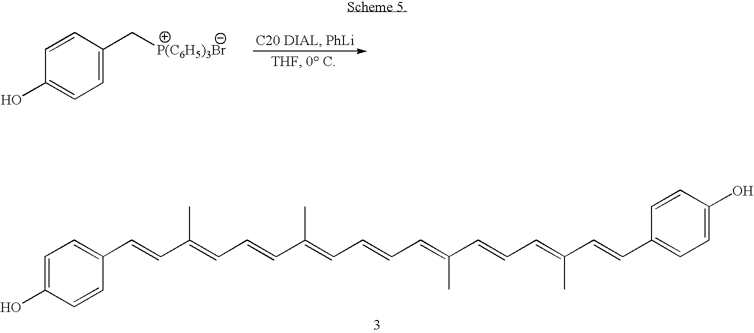

### Example 9

### Preparation of 4

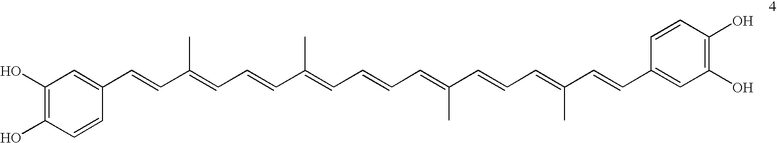

To a suspension of commercially available 3,4-dihydroxybenzyl alcohol (100 mg, 0.714 mmol) in acetonitrile (3 ml) and MeOH (0.5 ml) was added PPh₃-HBr (245 mg, 0.714 mmol) (Scheme 6). The reaction was refluxed for 12 h, after which the solution was cooled down to room temperature and concentrated down. The product was re-suspended in MeOH (0.5 ml) and diluted with acetonitrile. The product that precipitated out was filtered and washed with ice-cold acetonotrile. After repeating the procedure, afforded 166 mg (50% yield) 3,4-(dihydroxybenzyl)triphenylphosphonium bromide. ¹H NMR (300 MHz, CD₃OD) δ: 7.91-7.59 ppm (15H, m), 6.60-6.57 ppm (1H, d, J=7.97 Hz), 6.40-6.39 ppm (1H, t, J=2.22 Hz), 6.30-6.26 ppm (1H, dt, J=8.10/2.49 Hz), 4.74-4.69 ppm (2H, d, J=14.16 Hz), 3.01-2.96 (2H, d, J=14.12 Hz).

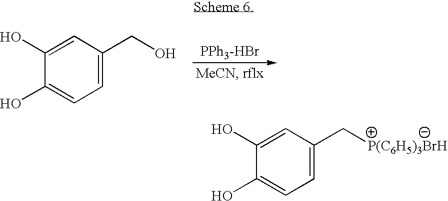

To a suspension of 3,4-(dihydroxybenzyl)triphenylphosphonium bromide (75 mg, 0.162 mmol) in tetrahydrofuran (5 ml) at 0° C., was added dropwise an ethereal solution of phenyllithium (20%, 1.62 mmol). The reaction was stirred for 10 min, added dichloromethane (200 ml) to quench excess base, and added dropwise a concentrated solution of C20 crocetindialdehyde (12 mg, 0.0406 mmol) in tetrahydrofuran (Scheme 7). The reaction was stirred at 0° C. for 10 min, warmed up to room temperature, and stirred for 30 min, at which time the solution was diluted with Et₂O, washed with aqueous NH₄Cl, brine, and water. The combined organic layers were then dried over MgSO₄, filtered and concentrated down to yield crude 2,3-dihydroxy phenolic carotenoid (4). LC/MS (APCI): 9.639 min (63% Area Under Curve), λₘₐₓ472 nm (100%), 504 nm (70%), 445 nm (90%), m/e: 508 (M) (10%), 509 (M+1) (15%), 510 (5%).

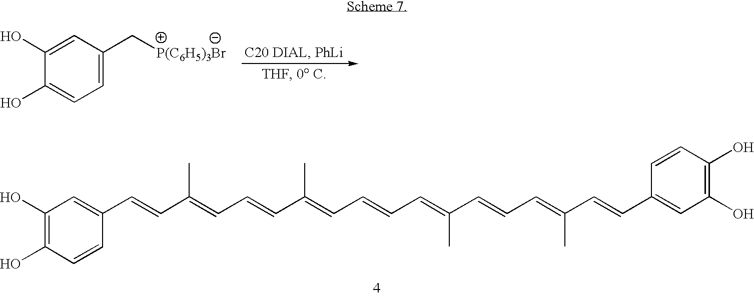

### Example 10

### Preparation of 5 and 6

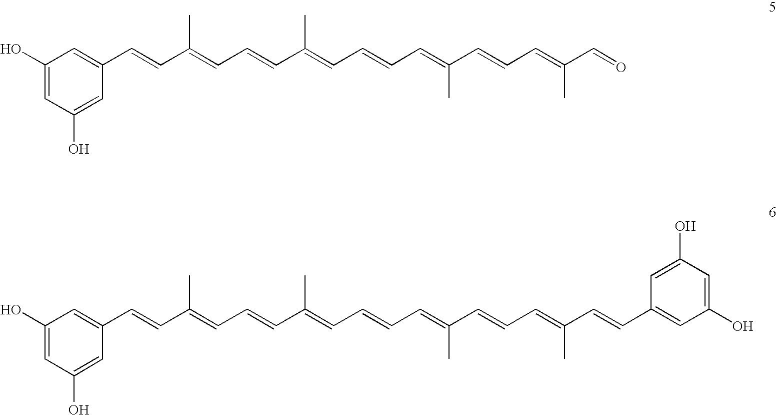

To a suspension of commercially available 3,5-dihydroxybenzyl alcohol (50 mg, 0.357 mmol) in acetonitrile (3 ml) was added PPh₃-HBr (122 mg, 0.357 mmol) (Scheme 8). The reaction was refluxed for 12 h, after which the solution was cooled down to 0° C. The product that precipitated out was filtered, washed with ice-cold acetonotrile, and dried to yield 180 mg (70% yield) 3,5-(dihydroxybenzyl)triphenylphosphonium bromide. MS (APCI): 3.093 min (>95% Area Under Curve), m/e: 385 (M-Br) (90%), 386 (20%), 279 (P(O)(C₆H₅)₃) (80%), 263 (P(C₆H₅)₃) (100%). ¹H NMR (300 MHz, d₆-DMSO): δ: 9.32 ppm (2H, s), 7.92-7.60 ppm (15H, m), 6.15-6.14 ppm (1H, d, J=2.09 Hz), 5.86-5.84 ppm (2H, t, J=2.24 Hz), 4.95-4.90 (2H, d, J=15.64 Hz).

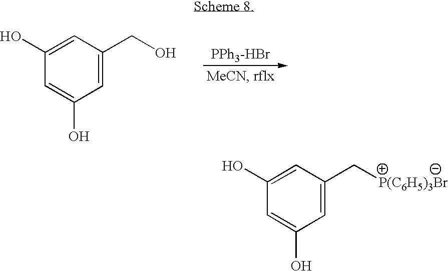

To a suspension of previously prepared 3,5-(dihydroxybenzyl)triphenylphosphonium bromide (65 mg, 0.140 mmol) in tetrahydrofuran (4 ml) at 0° C., was added drop wise an ethereal solution of phenyllithium (20%, 1.3 mmol). The reaction was stirred for 10 min, then added dropwise a concentrated solution of C20 crocetindialdehyde (13 mg, 0.0451 mmol) in tetrahydrofuran (Scheme 9). The reaction was stirred at 0° C. for 10 min, warmed up to room temperature, and stirred for 30 min. After concentrating down, a crude mixture of mono 2,4-dihydroxy phenolic carotenoid (5) and 2,4-dihydroxy phenolic carotenoid (6) was obtained. LC/MS (APCI): 9.087 min (14% Area Under Curve), λₘₐₓ430 nm (65%), 466 nm (100%), 490 nm (80%), m/e: 403 (M+1) (100%), 404 (30%), 8.656 min (40% Area Under Curve), λₘₐₓ472 nm (100%), 506 nm (88%), 448 nm (72%), m/e: 508 (M) (5%), 509 (M+1) (20%), 510 (10%).

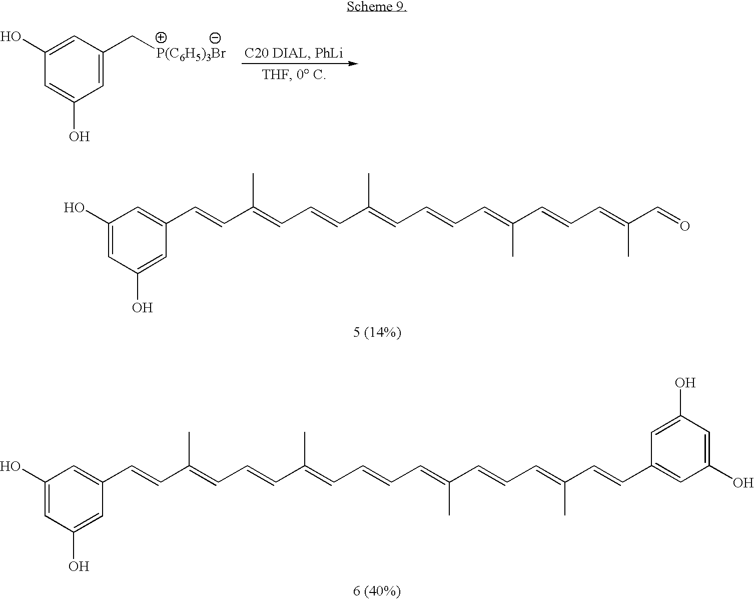

### Example 11

### Preparation of 7

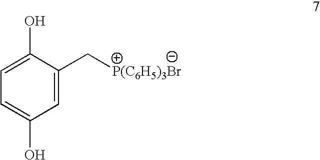

To a solution of 2,5-dihydroxybenzaldehyde (200 mg, 1.448 mmol) in tetrahydrofuran (4.5 ml) and water (150 μl) was added under stirring NaBH₄ (54 mg, 1.448 mmol) (Scheme 10). The rx mix was stirred at room temperature for 5 min, at which time the suspension was added solid NH₄Cl and water (0.5 ml), and stirred for 5 min. The suspension was diluted with tetrahydrofuran (THF), filtered, and concentrated down. The product was re-suspended in EtOH, filtered and concentrated down to yield 110 mg (54% yield) crude material. ¹H NMR (300 MHz, d₆-DMSO) δ: 7.72 (2H, s), 5.83-5.82 (1H, d, J=2.54 Hz), 5.67-5.64 (1H, d, J=8.48 Hz), 5.53-5.49 (1H, dd, J=2.79/8.46 Hz), 3.49 ppm (2H, s).

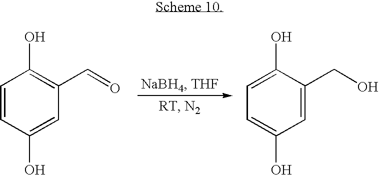

To a suspension of 2,5-dihydroxybenzyl alcohol (55 mg, 0.392 mmol) in acetonitrile (3 ml) and methanol (0.5 ml) was added PPh₃-HBr (135 mg, 0.392 mmol) (Scheme 11). The reaction was refluxed for 4 h, after which the solution was cooled down to 0° C., filtered, and concentrated down to yield 56 mg (31%) 2,5-(dihydroxybenzyl)triphenylphosphonium bromide (7). ¹H NMR (300 MHz, CD₃OD) δ: 7.90-7.60 ppm (15H, m), 6.63-6.59 ppm (1H, dt, J=8.72/2.70 Hz), 6.51-6.48 (1H, d, J=8.75 Hz), 6.38-6.36 ppm (1H, t, J=2.67), 4.71-4.63 ppm (2H, d, J=14.25 Hz), 3.026-2.98 ppm (2H, d, J=14.14 Hz).

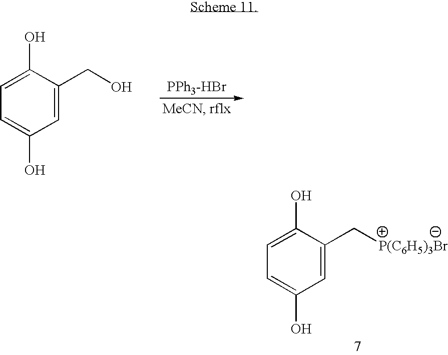

### Example 12

### Preparation of 8

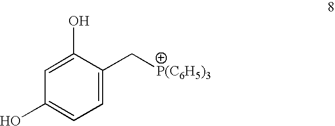

To a solution of 2,4-dihydroxybenzaldehyde (200 mg, 1.448 mmol) in tetrahydrofuran (4.5 ml) and water (150 μl) was added wider stirring NaBH₄ (54 mg, 1.448 mmol) (Scheme 12). The rx mix was stirred at for 5 min, at which time the suspension was added solid NH₄Cl and water (0.5 ml), and stirred for 5 min. The suspension was diluted with THF, filtered, and concentrated down to yield crude material. ¹H NMR (300 MHz, d₆-DMSO) δ: 8.24 (1H, s), 8.16 ppm (1H, s), 6.12-6.09 ppm (1H, d, J=8.16 Hz), 5.36-5.35 ppm (1H, d, J=1.68 Hz), 5.30-5.27 ppm (1H, dd, J=8.19/1.64 Hz), 3.47-3.45 ppm (2H, d, J=5.13 Hz).

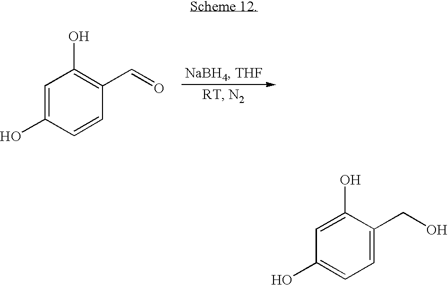

To a suspension of previously prepared crude 2,4-dihydroxybenzyl alcohol (130 mg, 0.926 mmol) in acetonitrile (3 ml) and methanol (0.5 ml) was added PPh₃-HBr (318 mg, 0.926 mmol) (Scheme 13). The reaction was refluxed for 5 h, after which time the solution was cooled down to 0° C., filtered, and concentrated down to yield 129 mg (30%) 2,4-(dihydroxybenzyl)triphenylphosphonium bromide (8). ¹H NMR (300 MHz, CD₃OD) δ: 7.89-7.59 ppm (15H, m), 6.68-6.64 ppm (1H, dd, J=8.17/2.69 Hz), 6.16-6.15 (1H, d, J=1.84 Hz), 6.14-6.11 ppm (1H, dd, J=8.22/2.36), 4.47-4.62 ppm (2H, d, J=13.23 Hz) 3.34 ppm (2H, s).

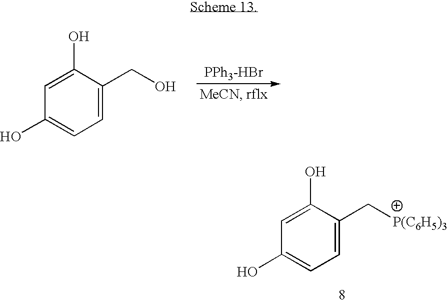

### Example 13

### Preparation of 9

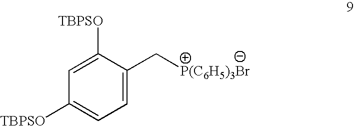

To a solution of commercially available 2,4-dihydroxybenzaldehyde (500 mg, 3.62 mmol) in N,N-dimethylformamide (DMF) (10 ml) was added in the following order, tert-butyldiphenylchlorosilane (TBPSCI) (2.3 ml, 7.96 mmol) and imidazole (542 mg, 7.96 mmol). The suspension was stirred at room temperature for 15 min and added 4-dimethyl amino pyridine (DMAP) (177 mg, 1.45 mmol) (Scheme 14). The rx mix was stirred at room temperature for 5 h at which time the mix was diluted with Et₂O and quenched with NH₄Cl. The organic phase was washed with aqueous NH₄Cl and brine. The combined organic phase was dried over MgSO₄, filtered, and concentrated down to yield crude product. After purification on a silica flash column with hexane/Et₂O (8:2), 1% triethylamine as eluent system, was afforded 350 mg (31% yield) TBPS-protected 2,4-dihydroxybenzaldehyde. MS (APCI): 14.962 min (>90% Area Under Curve), m/e: 615 (M+1) (100%), 616 (50%), 617 (20%). ¹H NMR (300 MHz, CDCl₃) δ: 10.51 ppm (1H, s), 7.61-7.55 ppm (1H, d, J=8.59 Hz), 7.49-7.15 ppm (20H, m), 6.37-6.34 (1H, d, J=8.51 Hz), 5.94 ppm (1H, s), 1.00 ppm (9H, s), 0.86 ppm (9H, s).

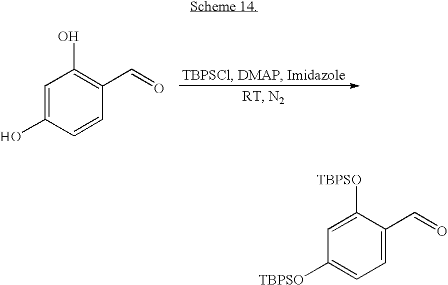

A solution of TBPS-protected 2,4-dihydroxybenzaldehyde (1.2 g, 1.9 mmol) was cooled down to −78° C. and added drop wise a solution of DIBAL (20% in toluene, 3.62 mmol) (Scheme 15). The rx mix was stirred at −78° C. for 1 h, at which time the suspension was added water (2.5 ml), warmed up to room temperature and added solid NaHCO₃ and Et₂OAc (19 ml). This mix was stirred at room temperature for 30 min, dried over MgSO₄, filtered and concentrated down to yield 865 mg crude material. Purification on a silica flash column with hexane/Et₂O (7:3), 1% triethylamine as eluent system, afforded 604 mg (54%) TBPS-protected 2,4-dihydroxybenzyl alcohol. MS (APCI): 12.874 min (95% Area Under Curve), m/e: 617 (M+1) (100%), 618(60%), 619(20%). ¹H NMR (300 MHz, CDCl₃) δ: 7.48-7.13 ppm (20H, m), 6.99-6.96 (1H, d, J=8.26 Hz), 6.28-6.26 ppm (1H, d, J=8.27 Hz), 6.00 ppm (1H, s), 4.66-4.64 ppm (2H, d, J=5.78 Hz), 1.00 ppm (9H, s), 0.84 ppm (9H, s).

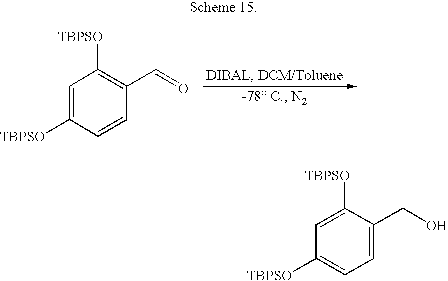

To a suspension of TBPS-protected 2,4-dihydroxybenzyl alcohol (212 mg, 0.343 mmol) in acetonitrile (5 ml) was added PPh₃-HBr (112 mg, 0.326 mmol) (Scheme 16). The reaction was refluxed for 1.5 h, after which the solution was cooled down to room temperature. The solution was concentrated down to yield 366 mg TBPS-protected 2,4-(dihydroxybenzyl)triphenylphosphonium bromide (9). MS (APCI): 9.903 min (>80% Area Under Curve), m/e: 861 (M-Br) (20%), 279 (P(O)(C₆H₅)₃) (20%), 263 (P(C₆H₅)₃) (100%).

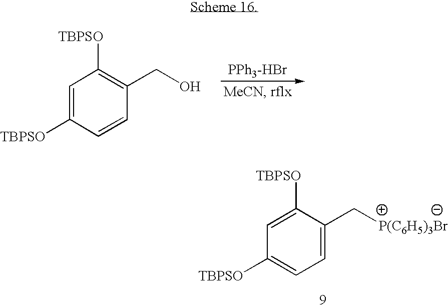

### Example 14

### Preparation of 10

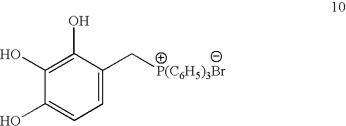

To a solution of 2,3,4-trihydroxybenzaldehyde (150 mg, 0.974 mmol) in tetrahydrofuran (3 ml) and water (100 μl) was added under stirring NaBH₄ (37 mg, 0.974 mmol) (Scheme 17). The rx mix was stirred at room temperature for 5 min, at which time the suspension was added MeOH (2 ml), water (1 ml) and solid NH₄Cl to neutral pH. The suspension was stirred for 5 min, diluted with EtOH, and concentrated down. The solid was re-suspended in a mixture of EtOH and THF (6:4), the impurities were filtered off, and the residual concentrated down to yield crude material. ¹H NMR (300 MHz, CD₃OD) δ: 6.54-6.52 ppm (1H, d, J=8.54 Hz), 6.29-6.26 ppm (1H, d, J=8.11 Hz), 4.53 ppm (3H, s), 3.27 ppm (2H, s).

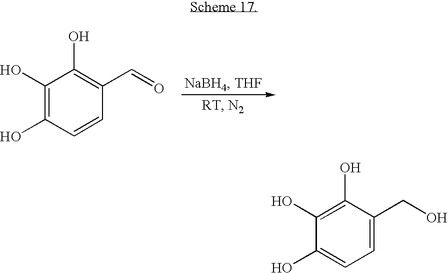

To a suspension of previously prepared crude 2,3,4-trihydroxybenzyl alcohol (145 mg, 0.935 mmol) in acetonitrile (3 ml) and THF (0.5 ml) was added PPh₃-HBr (320 mg, 0.935 mmol) (Scheme 18). The reaction was refluxed for 7 h, after which time the solution was cooled down to 0° C., filtered, and concentrated down. Re-suspended in acetonitrile, filtered off the impurities, and concentrated down to yield 183 mg (41%) 2,3,4-(trihydroxybenzyl)triphenylphosphonium bromide (10). ¹H NMR (300 MHz, d₆-DMSO) δ: 9.33 ppm (1H, s), 8.71 ppm (1H, s), 8.46 ppm (1H), 7.90-7.39 ppm (15H, m), 6.12-6.09 ppm (1H, d, J=8.41 Hz), 6.04-6.01 (1H, dd, J=8.44/2.65 Hz), 4.80-4.76 ppm (2H, d, J=13.99).

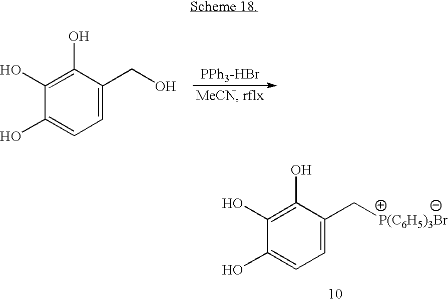

### Example 15

### Preparation of 11

To a suspension of crude TES-protected 2,3,4-trihydroxybenzyl alcohol (150 mg, 0.300 mmol) in acetonitrile (5 mL) and Et₂O (0.5 ml) was added PPh₃-HBr (83 mg, 0.241 mmol) (Scheme 19). The reaction was refluxed for 2 h, after which the solution was cooled down to room temperature. The solution was concentrated down to yield crude TES-protected 2,3,4-(trihydroxybenzyl)triphenylphosphonium bromide (11). MS (APCI): 9.410-10.064 min, (>45% Area Under Curve), m/e: 629 (M-Br-TES) (20%), 630 (10%), 631 (5%), 279 (P(O)(C₆H₅)₃) (35%), 263 (P(C₆H₅)₃) (100%).

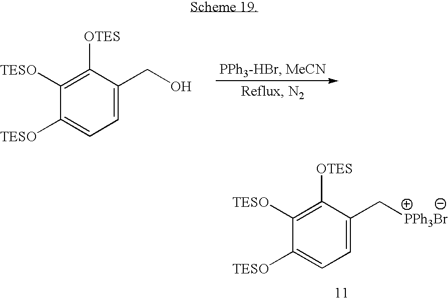

### Racemic Astaxanthin (12)

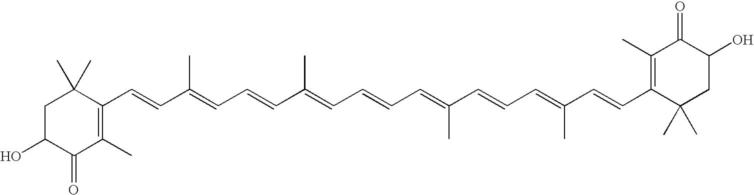

LC; ultraviolet-visible (UV-VIS); MS: 11.78 min (95%); 478 nm (100%); m/e: 597 (M+H) (100%), 596 (M) (17%), 363 (9%).

### Example 16

### Preparation of Astacene (13)

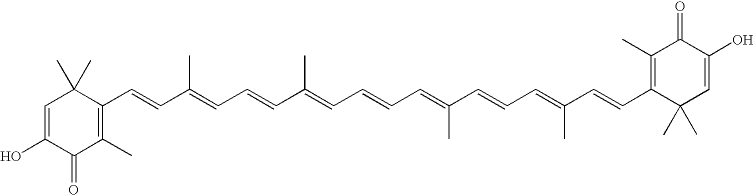

To a solution of 12 (1.68 mmol, 1.0 g) in toluene/methanol (20 mL/40 mL) was added methanolic sodium methoxide (25% wt; 16.8 mmol, 3.8 μL). The solution was stirred at rt overnight, then concentrated in vacuo. The residue was suspended in tetrahydrofuran (20 mL), filtered, then concentrated down to yield 13 (87%; 1.46 mmol, 0.86 g) as a dark red solid. LC; UV-VIS; MS: 12.07 min (95%); 484 nm (100%); m/e: 593 (M+H) (100%), 592 (M) (9%), 391 (6%).

### Example 17

### Preparation of Roserythrin (14)

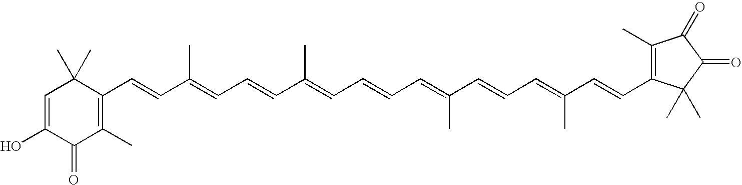

To a solution of 13 (1.10 mmol, 0.65 g) in acetone (70 mL) was added MnO₂ (85% wt; 16.5 mmol, 1.43 g). The mixture was stirred at room temperature overnight, then concentrated in vacuo. The residue was suspended in methanol (20 mL), filtered, and concentrated down to yield 14 (27%), violerythrin (16%), and unreacted astacene (51%). LC; UV-VIS; MS: 11.22 min (27%); 528 nm (100%); m/e: 579 (M+H) (100%), 578 (M) (12%), 282 (78%).

### Example 18

### Preparation of Violerythrin (15)

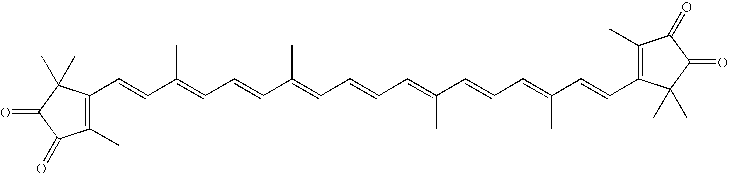

To a solution of 13 (1.10 mmol, 0.65 g) in acetone (70 mL) was added MnO₂ (85% wt; 99 mmol, 8.6 g). The mixture was stirred at rt overnight, then concentrated in vacuo. The residue was suspended in methanol (20 mL), filtered, concentrated down, then resuspended in dichloromethane (20 mL), filtered, and concentrated down to yield 15 (47%; 0.52 mmol, 0.29 g) as a black-purple solid. LC; UV-VIS; MS: 10.41 min (4%); 368 nm (12%), 571 nm (100%); m/e: 565 (M+H) (100%), 564 (M) (8%), 421 (11%); 10.61 min (86%); 368 nm (13%), 571 nm (100%); m/e: 565 (M+H) (100%), 564 (M) (7%), 421 (11%); 11.03 min (10%); 368 nm (12%), 571 nm (100%); m/e: 565 (M+H) (100%), 564 (M) (5%), 279 (10%).

### Example 19

### Preparation of Racemic Actinioerythrol (16)

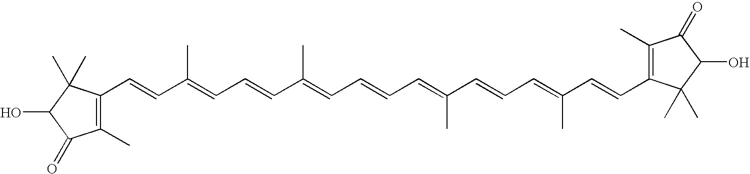

To a solution of 15 (0.018 mmol, 0.010 g) in dichloromethane/methanol (2 mL/4 mL) at ice bath temp was added dropwise a methanolic solution of NaBH₄ (0.1% vol; 0.004 mmol, 0.15 mL). The solution was stirred for 5 min, then 40% aqueous ethanol (3 mL) was added, and the mixture concentrated down to yield 16 (89%; 0.016 mmol, 0.009 g) as a red solid. LC; UV-VIS; MS: 8.82 min (14%); 325 nm (20%), 503 nm (100%), 530 nm (89%); m/e: 569 (M+H) (13%), 568 (M) (41%), 567 (100%), 279 (8%); 10.22 min (86%); 325 nm (18%), 503 nm (100%), 530 nm (90%); m/e: 569 (M+H) (15%), 568 (M) (44%), 567 (100%), 279 (9%).

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1. A chemical compound having the structure: 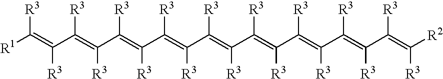
wherein each R³ is independently hydrogen or methyl, and wherein each R¹ and R² are independently: 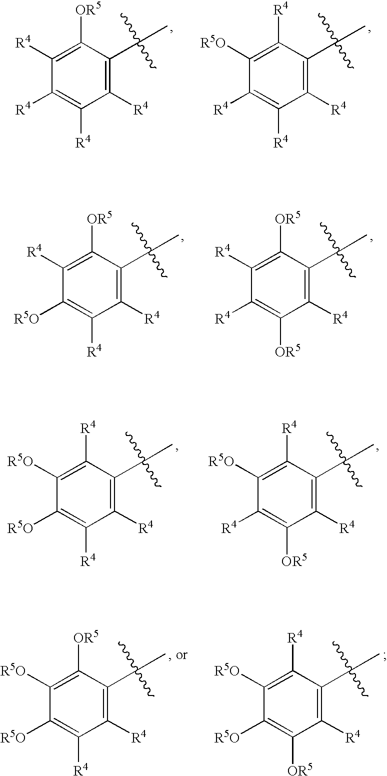
wherein each R⁴ is independently hydrogen or methyl; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(R⁶)₃; —C(NR⁶)-aryl-N⁺(R⁶)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

2. The chemical compound of claim 1, wherein the chemical compound has the structure: 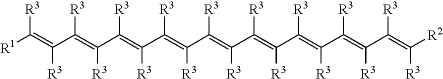
wherein each R³ is independently hydrogen or methyl, and wherein each R¹ and R² are independently: 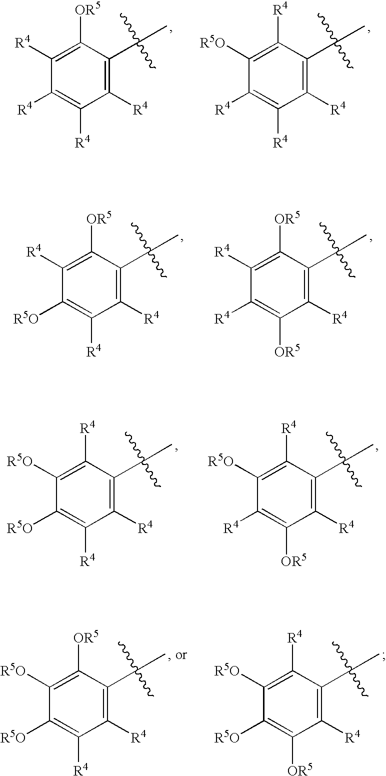
wherein each R⁴ is independently hydrogen or methyl; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻;
alkyl-N(R 6)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; wherein R⁹ is a co-antioxidant; and wherein n is 1 to 9.

3. The chemical compound of claim 1, wherein the chemical compound has the structure: 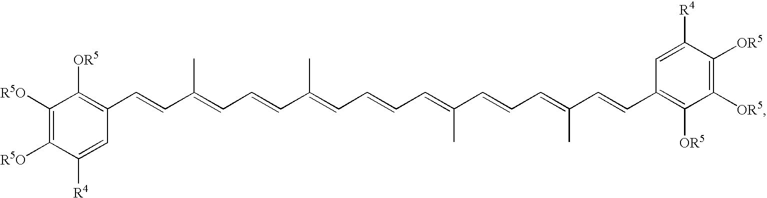
wherein each R⁴ is independently hydrogen or methyl; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

4. The chemical compound of claim 1, wherein the chemical compound has the structure: 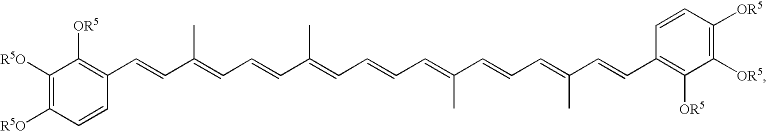
wherein each R⁴ is independently hydrogen or methyl; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R 6)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

5. The chemical compound of claim 1, wherein the substituent —OR⁵ is: 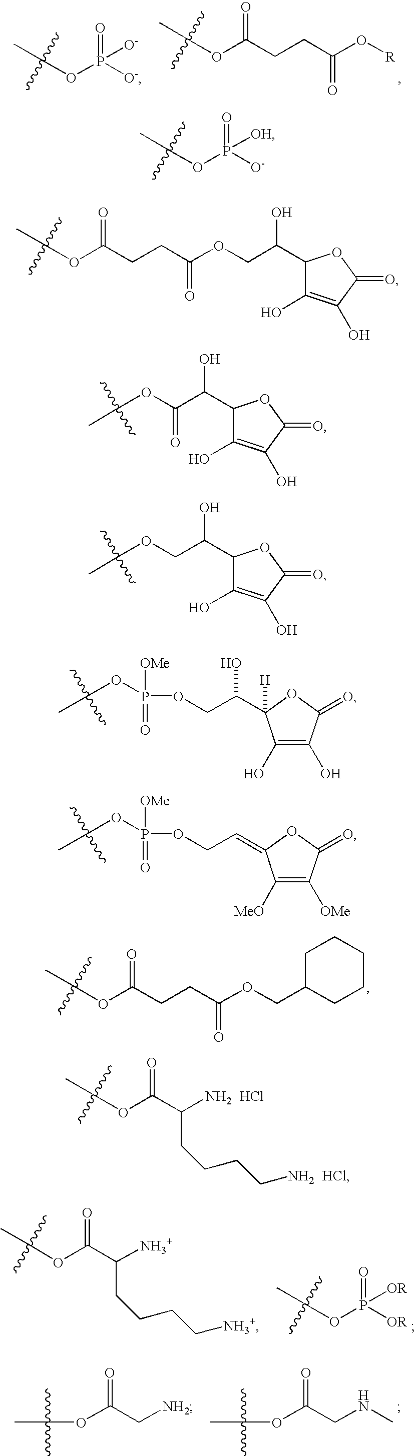 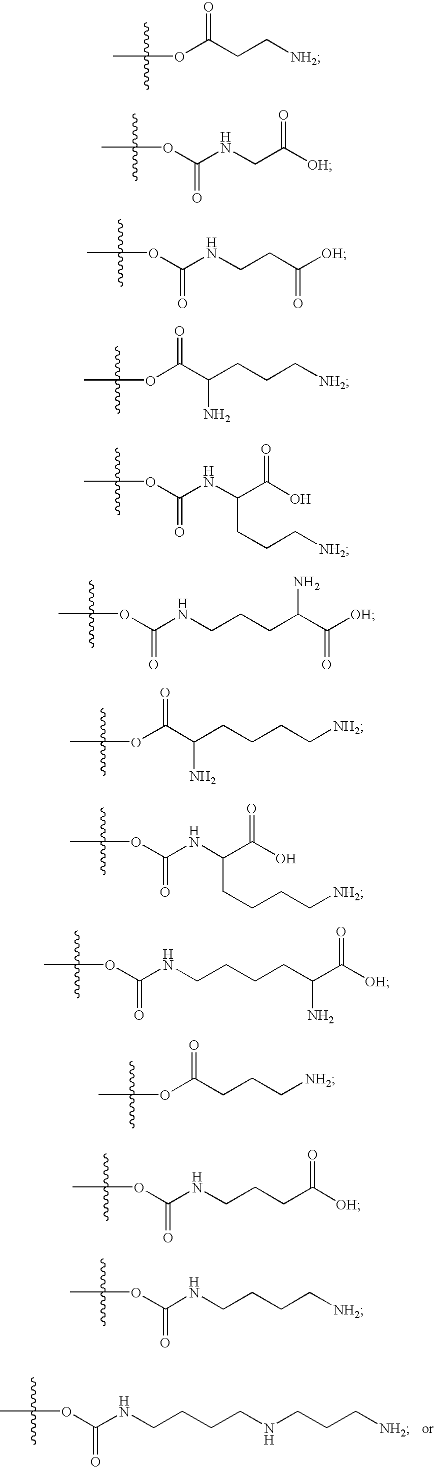 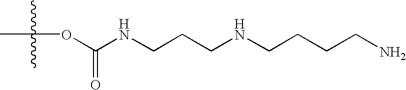
or pharmaceutically acceptable salts thereof and wherein each R is independently H, alkyl, aryl, benzyl, Group IA metal, or co-antioxidant.

6. The chemical compound of claim 1, wherein the substituent —OR⁵ is: 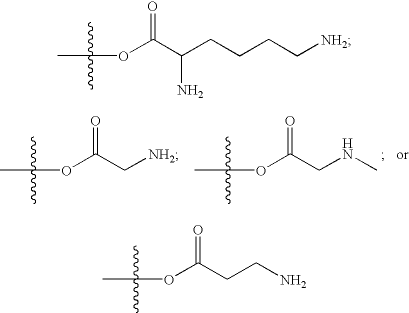
or pharmaceutically acceptable salts thereof.

7. The chemical compound of claim 1, wherein R⁵ is an amino acid, amino acid derivative, or amino acid analog.

8. The chemical compound of claim 1, wherein the chemical compound has the structure:

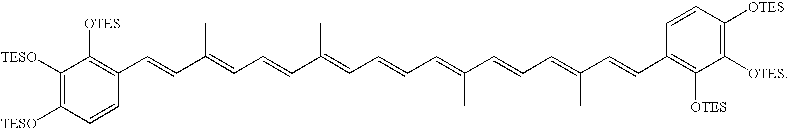

9. The chemical compound of claim 1, wherein the chemical compound has the structure:

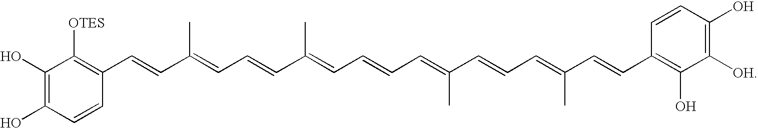

10. The chemical compound of claim 1, wherein the chemical compound has the structure:

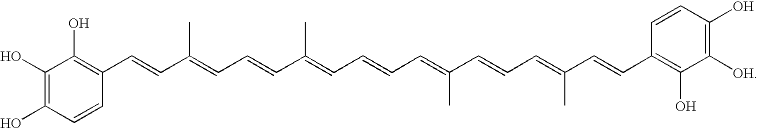

11. The chemical compound of claim 1, wherein the co-antioxidant comprises Vitamin C, Vitamin C analogs, Vitamin C derivatives, Vitamin E, Vitamin E analogs, Vitamin E derivatives, polyphenolics, flavonoids, flavonoid derivatives, or flavonoid analogs.

12. The chemical compound of claim 11, wherein the flavonoids comprise quercetin, xanthohumol, isoxanthohumol, or genistein.

13. The chemical compound of claim 11, wherein the polyphenolics comprise resveratrol.

14-26. (canceled)

27. A chemical compound having the structure: 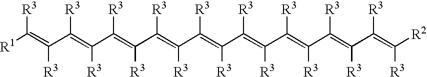
wherein each R³ is independently hydrogen or methyl, and wherein each R¹ and R² are independently: 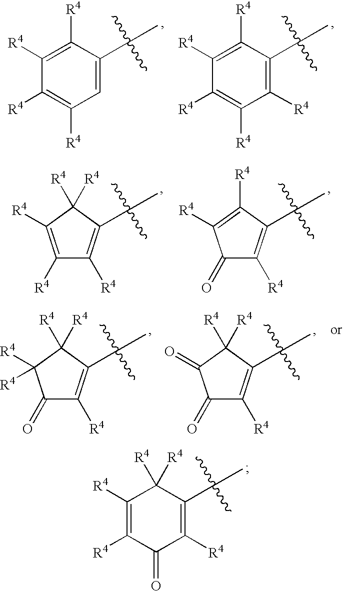
wherein each R⁴ is independently hydrogen, methyl, —OH, or —OR⁵ wherein at least one R⁴ group is —OR⁵; wherein each R⁵ is independently: hydrogen, alkyl; aryl; -alkyl-N(R⁶)₂; -aryl-N(R 6)₂; -alkyl-N⁺(R 6)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R 6)₂; —C(NR⁶)-aryl-N(R 6)₂; —C(NR⁶)-alkyl-N⁺(R⁶)₃; —C(NR⁶)-aryl-N⁺(R 6)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein at least one R⁵ is -alkyl-N(R⁶)₂; -aryl-N(R 6)₂; -alkyl-CO₂H; -aryl-CO₂H; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

28. The chemical compound of claim 27, wherein the chemical compound has the structure: 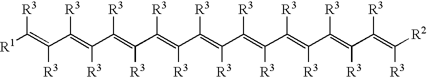
wherein each R³ is independently hydrogen or methyl, and wherein each R¹ and R² are independently: 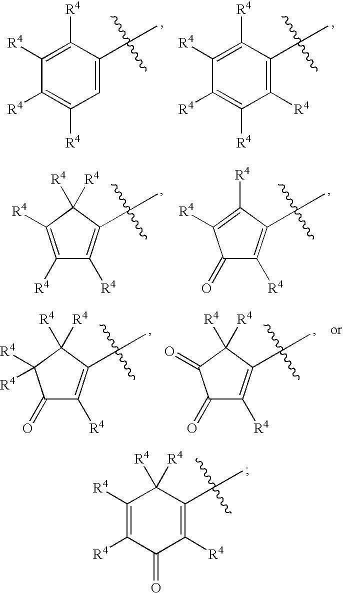
wherein each R⁴ is independently hydrogen, methyl, —OH, or —OR⁵ wherein at least two R⁴ groups are —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; -C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

29. The chemical compound of claim 27, wherein the chemical compound has the structure: 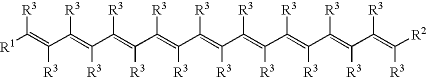
wherein each R³ is independently hydrogen or methyl, and wherein each R¹ and R² are independently: 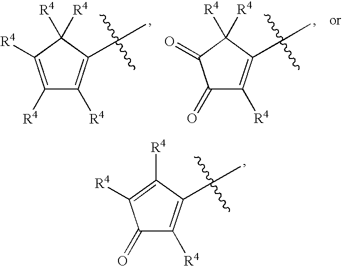
wherein each R⁴ is independently hydrogen, methyl, —OH, or —OR⁵ wherein at least one R⁴ groups is —OR⁵; wherein each R⁵ is independently: hydrogen, alkyl; aryl; -alkyl-N(R⁶)₂; -aryl-N(R 6)₂; -alkyl-N⁺(R 6); -aryl-N⁺(R⁶)₃; -alkyl-CO₂R⁷; -aryl-CO₂R⁷; -alkyl-CO₂⁻; -aryl-CO₂⁻; —C(O)-alkyl-N(R 6)₂; —C(O)-aryl-N(R 6)₂; —C(O)-alkyl-N⁺(R 6)₃; —C(O)-aryl-N⁺(R 6)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein at least one R⁵ is -alkyl-N(R⁶)₂; -aryl-N(R⁶)₂; -alkyl-CO₂H; -aryl-CO₂H; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

30. The chemical compound of claim 27, wherein the chemical compound has the structure: 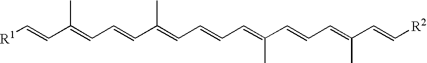
wherein each R¹ and R² are independently: 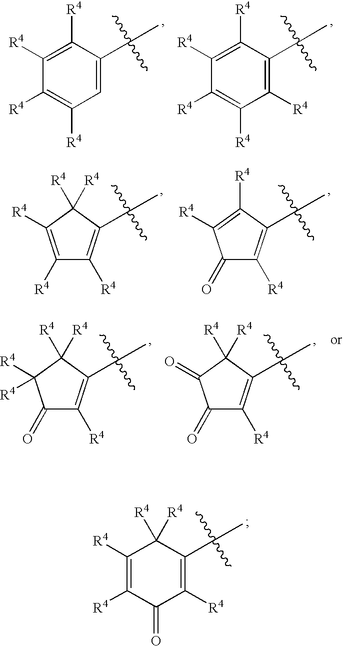
wherein each R⁴ is independently hydrogen, methyl, —OH, or —OR⁵ wherein at least two R⁴ groups are —OR⁵; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-alkyl-CO₂⁻; -alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein at least one R⁵ is -alkyl-N(R⁶)₂; -aryl-N(R⁶)₂; -alkyl-CO₂H; -aryl-CO₂H; —P(O)(OR⁷)₂; —S(O)(OR⁷)₂; SiR⁶₃; an amino acid; a peptide, a carbohydrate; —C(O)—(CH₂)ₙ—CO₂R⁸; a nucleoside residue, or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

31. (canceled)

32. (canceled)

33. The chemical compound of claim 27, wherein the substituent —OR⁵ is: 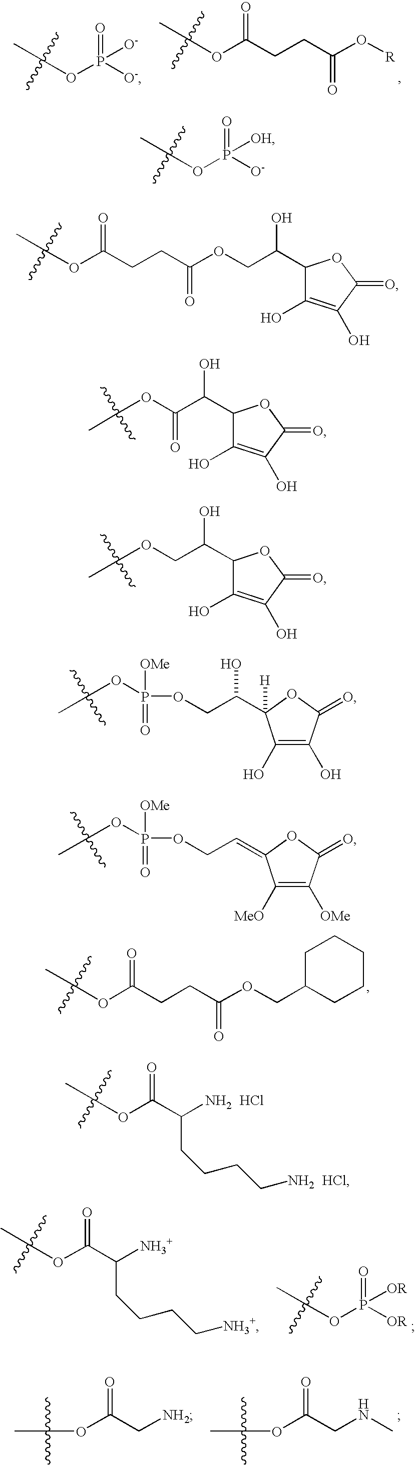 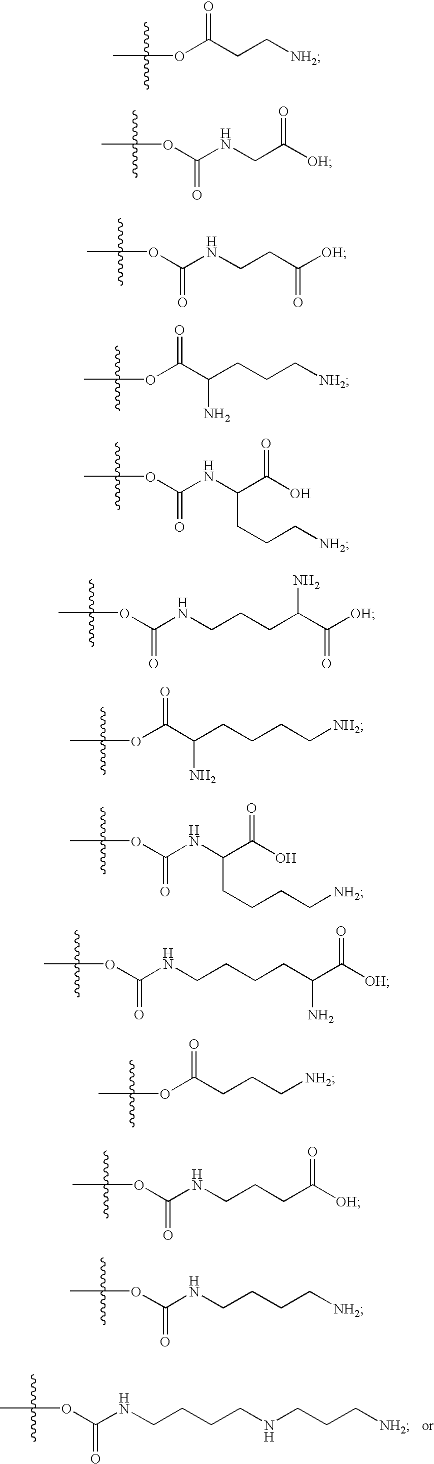 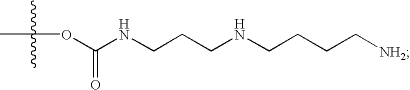
or pharmaceutically acceptable salts thereof and wherein each R is independently H, alkyl, aryl, benzyl, Group IA metal, or co-antioxidant.

34. The chemical compound of claim 27, wherein the substituent —OR⁵ is: 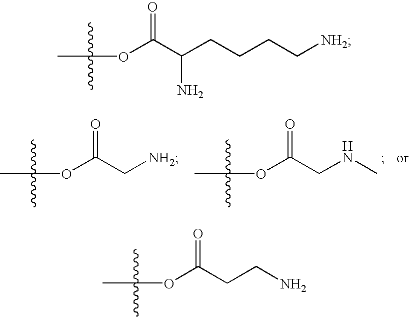
or pharmaceutically acceptable salts thereof.

35-56. (canceled)

57. A method of inhibiting and/or ameliorating a disease associated with reactive oxygen species and/or other radical and non-radical species comprising administering to a subject a carotenoid analog, carotenoid derivative, or pharmaceutically acceptable derivative of a carotenoid analog or a carotenoid derivative having the structure: 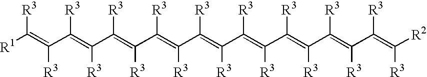
wherein each R³ is independently hydrogen or methyl, and wherein each R¹ and R² are independently: 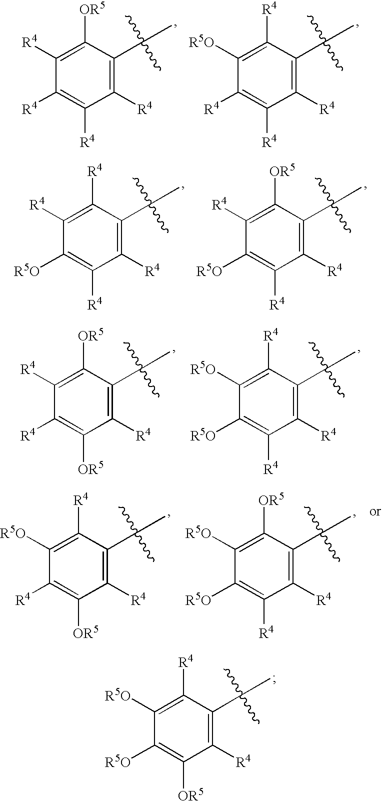
wherein each R⁴ is independently hydrogen or methyl; wherein each R⁵ is independently: hydrogen, alkyl; aryl; -alkyl-N(R⁶)₂; -aryl-N(R⁶)₂; -alkyl-N⁺(R⁶)₃; -aryl-N⁺(R⁶)₃; -alkyl-CO₂R⁷; -aryl-CO₂R⁷; -alkyl-CO₂⁻; -aryl-CO₂⁻; —C(O)-alkyl-N(R⁶)₂; —C(O)-aryl-N(R⁶)₂; —C(O)-alkyl-N⁺(R⁶)₃; —C(O)-aryl-N⁺(R⁶)₃; —C(O)-alkyl-CO₂R⁷; —C(O)-aryl-CO₂R⁷; —C(O)-alkyl-CO₂⁻; —C(O)-aryl-CO₂⁻; —C(NR⁶)-alkyl-N(R⁶)₂; —C(NR⁶)-aryl-N(R⁶)₂; —C(NR⁶)-alkyl-N⁺(R⁶)₃; —C(NR⁶)-aryl-N⁺(R⁶)₃; —C(NR⁶)-alkyl-CO₂R⁷; —C(NR⁶)-aryl-CO₂R⁷; —C(NR⁶)-alkyl-CO₂⁻; —C(NR⁶)-aryl-CO₂⁻; —C(NR⁶)-alkyl-N(R⁶)-alkyl-N(R⁶)₂; —C(O)—OR⁷; —P(O)(OR⁷)₂; —S(O)(OR⁷)₂; —SiR⁶₃; —C(O)—[C₆-C₂₄ saturated hydrocarbon]; —C(O)—[C₆-C₂₄ monounsaturated hydrocarbon]; —C(O)—[C₆-C₂₄ polyunsaturated hydrocarbon]; an amino acid; a peptide; a carbohydrate; a nucleoside reside; a Group IA metal or a co-antioxidant; wherein R⁶ is hydrogen, alkyl, or aryl; wherein R⁷ is hydrogen, alkyl, aryl, benzyl, Group IA metal or a co-antioxidant; wherein R⁸ is hydrogen; alkyl; aryl; —P(O)(OR⁷)₂; —S(O)(OR⁷)₂; an amino acid; a peptide, a carbohydrate; a nucleoside, or a co-antioxidant; and wherein n is 1 to 9.

58. (canceled)
